(19) United States
(12) Patent Application Publication
(10) Pub. No.: US 2023/0087306 A1
(43) Pub. Date: Mar. 23, 2023

(54) UNCONJUGATED PLGA NANOPARTICLES IN THE TREATMENT OF ALZHEIMER'S DISEASE

(71) Applicant: The Governors of the University of Alberta, Edmonton (CA)

(72) Inventors: Satyabrata KAR, Edmonton (CA); Bibin Gnanadhason ANAND, Edmonton (CA); Qi WU, Edmonton (CA); Karthivashan GOVINDARAJAN, Edmonton (CA)

(21) Appl. No.: 17/873,751

(22) Filed: Jul. 26, 2022

Related U.S. Application Data
(60) Provisional application No. 63/203,547, filed on Jul. 27, 2021.

(51) Int. Cl. A61K 9/16; A61P 25/28

(52) U.S. Cl. CPC A61K 9/1647; A61P 25/28

(57) ABSTRACT

The present disclosure relates generally to unconjugated PLGA nanoparticles in the treatment of Alzheimer's Disease. The present disclosure provides a method of treating a subject having Alzheimer's Disease or a tauopathy or suspected of having Alzheimer's Disease or a tauopathy, the method includes administering a therapeutically effective amount of poly(D,L-lactide-co-glycolide) (PLGA) to the subject.

### FIELD

The present disclosure relates generally to unconjugated PLGA nanoparticles in the treatment of Alzheimer's Disease.

### BACKGROUND

Alzheimer's disease (AD) is an unremitting neurodegenerative disorder characterized by a gradual loss of memory followed by deterioration of higher cognitive functions such as language, praxis and judgement. AD afflicts ˜7% of the population over 65 years of age and its prevalence doubles every 5 years thereafter (1-3). Etiologically, most AD cases are sporadic, whereas only a minority (<10%) of cases segregate with mutations in three genes; amyloid precursor protein (APP) on chromosome 21, presenilin 1 (PSEN1) on chromosome 14 and PSEN2 on chromosome 1. Other factors that increase the risk of AD include age, head injury, stress and inheritance of apolipoprotein E4 genotype (4-7). The neuropathological features associated with AD include the presence of intracellular neurofibrillary tangles, extracellular neuritic plaques and severe neuronal loss in selected brain regions. Structurally, tangles are comprised of hyperphosphorylated tau protein, whereas neuritic plaques contain a central deposit of δ-amyloid (Aβ) peptides generated from APP by successive cleavage via β-secretase and tetrameric γ-secretase complex (2, 8, 9). Pathological changes that characterize AD, together with the constitutive production of Aβ in the normal brain (1, 10, 11), indicate that an overproduction and/or a lack of clearance may lead to increased Aβ levels which, in turn, contribute to neuronal loss and development of AD. The brain regions affected in AD include the basal forebrain, hippocampus and neocortex, whereas striatum and cerebellum are spared (8, 9). Despite extensive research, it remains unclear how altered levels/function of Aβ can target selective neuronal populations in AD brains.

Earlier studies reported that Aβ peptides containing 39-43 amino acid residues are generated from APP, but the two most prevalent isoforms found in the brain are Aβ₁₋₄₀ and Aβ₁₋₄₂(1, 10). Monomeric peptides present in pM-nM concentrations are known to regulate a variety of functions including synaptic activity and release of neurotransmitters in the normal brain. While Aβ₁₋₄₂ constitutes ˜10% of the total Aβ secreted from cells, it aggregates faster and is more toxic to neurons than Aβ₁₋₄₀(12-16). Mounting evidence suggests that conversion of Aβ from its soluble monomer into various aggregated states (i.e., soluble oligomers, protofibrils and mature fibrils) may underlie the cause of AD as these conformations can differentially render neurons vulnerable to death. Thus, preventing the conversion of the Aβ monomer into toxic aggregates is a promising strategy for AD treatment (15, 17, 18).

At present, there is no effective treatment to arrest the progression of AD. The cholinesterase inhibitors (Donepezil, Galantamine and Rivastigmine) and the glutamate NMDA receptor antagonist memantine that have been approved for the treatment provide symptomatic relief for only a fraction of AD patients (18-20). A new treatment designed to remove Aβ plaques by passive immunization using anti-Aβ antibody Aducanumab (Biogen Inc.) was recently approved by the US Food and Drug Administration (FDA) for AD patients, but its potential benefits remain to be confirmed (21). Other strategies that are currently under development for treatment include novel inhibitors of cholinesterase, neuroprotective agents, drugs targeting Aβ metabolism and vaccination against Aβ peptide (22-24). One of the limiting factors, however, is the blood-brain barrier (BBB) preventing the penetration of the majority of drugs/agents into the brain (25, 26). Over the last decade, nanoparticles, which are engineered materials less than 100 nm in diameter with unique physiochemical properties, have been explored extensively as an area of novel therapeutics to overcome the protective BBB. The drugs/molecules are easily dissolved, entrapped and encapsulated within and/or functionalized to the surface of the nanoparticles which can then penetrate the BBB, allowing an efficient accumulation of the drug at the targeted site (25-28). The use of biodegradable materials for nanoparticle preparations allows sustained release of the drug at the targeted site over a period of days or even weeks after administration (29). Strategies utilizing nanoparticles that are in different stages of development for the treatment of AD include targeted delivery of cholinesterase inhibitors (donepezil, tacrine, rivastigmine, galantamine), phytochemicals (epigallocatechin-3-gaggate—a polyphenolic compound obtained from green tea; resveratrol—a polyphenolic found in grapes and red wine; curcumin—a polyphenolic compound isolated from the Curcuma lunga turmertic plant; ginsenoside Rg3—an active ingredient isolated from ginseng extracts; quercetin—a polyphenolic compound found in many fruits, vegetables and red wine), hormones (estradiol, melatonin), metal chelators (deferoxamine—an iron chelator; D-penicillamine—a copper chelator; clioquinol—a cupper/zinc chelator), antibodies and various drugs/agents (memantine—an NMDA receptor antagonist; selegiline—a selective monoamine oxidase-B inhibitor; pioglitazone—an agonist of the peroxisome proliferator-activated receptor; carbenoxolone—an extract from liquorice root used for the treatment of peptic ulcers; vitamin D-binding protein) to sequester Aβ peptide or to interfere with Aβ aggregation and toxicity (27, 28, 30-44).

Evidence suggests that the structure and properties of nanoparticles, especially the surface charge and hydrophobicity, play important role in the interactions between Aβ and nanoparticles (45). Considering the strong intrinsic hydrophobicity of Aβ peptide, the exposed hydrophobic domain of the nanoparticles will bind Aβ and inhibit the aggregation process, leading to decreased toxicity (46, 47). Indeed, it has been shown that nanoliposomes functionalized with cardiolipin or phosphatidic acid can reduce Aβ toxicity (48), whereas polymorphic nanoparticles conjugated with curcumin, donepezil or memantine can inhibit Aβ aggregation/toxicity under in vitro conditions and/or can decrease Aβ burden as well as cognitive deficits in animal models of AD (30-44, 49).

Interestingly, acidic poly(D,L-lactide-co-glycolide) (PLGA) nanoparticles which constitute a family of FDA- as well as European Medicine Agency-approved biodegradable polymers have been studied extensively as delivery vehicles for various drugs, peptides, proteins and other macromolecules. In fact, PLGA is currently in clinical use for delivery of many FDA-approved drugs in a variety of conditions (25, 50-52). Additionally, some recent studies have shown that certain drugs/agents (such as donepezil, memantine, curcumin, quercetin, selegiline) encapsulated within PLGA nanoparticles can have beneficial effects on cellular and/or animal models of AD (30-36, 41-44). PLGA is synthesized from two different monomers, the cyclic glycolic acid and lactic acid (50, 51). Depending on the ratio of lactide to glycolide used for the polymerization, different forms of PLGA can be obtained, which are readily hydrolyzed in the body to serve as normal physiological by-products of various metabolic pathways. The degradation rate of PLGA is related to the ratio of monomers used in production; PLGA polymer containing a 50:50 ratio of lactic and glycolic acids is hydrolyzed much faster than those containing higher proportions of either of the two monomers (53-58). Additionally, in vitro and in vivo studies testing the toxicity have demonstrated that PLGA exhibits satisfactory biocompatibility without any significant toxicity (59).

It is reported that PLGA can be internalized into the cells through fluid phase pinocytosis or clathrin-mediated endocytosis and then traffic to lysosomes where it reduces lysosomal pH, leading to improved degradative activity of the lysosomal enzymes. The magnitude of acidification induced by PLGA is larger in compromised lysosomes with elevated pH than at baseline levels, suggesting PLGA's potential implication in the treatment of various diseases associated with lysosomal dysfunction including AD (56, 58). An earlier study showed that native PLGA (without conjugation with any drug/agent) can attenuate death of cultured cells caused by the mitochondrial toxin 1-methyl-4-phenyl-1,2,3,6-tetrahydropyridine (i.e., MPTP—used in the development of an animal model of Parkinson Disease, PD) or genetic mutation associated with PD by restoring impaired lysosomal function. Intracerebral injection of PLGA can also attenuate MPTP-related neurodegeneration by rescuing the function of compromised lysosomes (59).

### SUMMARY

In one aspect there is provided a method of treating a subject having Alzheimer's Disease or a tauopathy or suspected of having Alzheimer's Disease or a tauopathy, comprising: administering a therapeutically effective amount of poly(D,L-lactide-co-glycolide) (PLGA).

In one aspect there is provided a method of treating a subject having Alzheimer's Disease or a tauopathy or suspected of having Alzheimer's Disease or a tauopathy, consisting of or essentially consisting of: administering a therapeutically effective amount of poly(D,L-lactide-co-glycolide) (PLGA).

In one example, the poly(D,L-lactide-co-glycolide) polymer contains a 50:50 ratio of lactic and glycolic acids.

In one example, said Alzheimer's Disease is preclinical Alzheimer's Disease, Mild cognitive impairment, or Alzheimer's dementia.

In one example, said tauopathy is frontotemporal dementia, Pick's disease, epilepsy, or chronic traumatic encephalopathy.

In one example, said administration comprises intracerebroventricular, intravenous, intranasal, or subcutaneous administration.

In one aspect there is provided a use of a therapeutically effective amount of poly(D,L-lactide-co-glycolide) (PLGA) for treating a subject having Alzheimer's Disease or a tauopathy or suspected of having Alzheimer's Disease or a tauopathy.

In one aspect there is provided a use of a therapeutically effective amount of poly(D,L-lactide-co-glycolide) (PLGA) in the manufacture of a medicament for treating a subject having Alzheimer's Disease or a tauopathy or suspected of having Alzheimer's Disease or a tauopathy.

In one example, the poly(D,L-lactide-co-glycolide) polymer contains a 50:50 ratio of lactic and glycolic acids.

In one example, said Alzheimer's Disease is preclinical Alzheimer's Disease, Mild cognitive impairment, or Alzheimer's dementia.

In one example, said tauopathy is frontotemporal dementia, Pick's disease, epilepsy, or chronic traumatic encephalopathy.

In one example, said PLGA is formulated for intracerebroventricular, intravenous, intranasal, or subcutaneous administration.

### BRIEF DESCRIPTION OF THE FIGURES

Embodiments of the present disclosure will now be described, by way of example only, with reference to the attached Figures.

FIG. 1. Oligomeric human Aβ₁₋₄₂-induced toxicity in primary mouse cortical neurons A & B: Histogram depicting dose-(FIG. 1A) and time-(FIG. 1B) dependent decrease in the viability of mouse cortical cultured neurons following treatment with oligomeric human Aβ₁₋₄₂ as revealed by MTT reduction. FIG. 1C; Histogram depicting neuronal toxicity over 48 h as detected using the LDH assay following 10 μM Aβ₁₋₄₂ treatment.

FIG. 1D & FIG. 1E: Photomicrographs of live/dead assay showing live and dead cells in control (D) and 24 h Aβ₁₋₄₂ (10 μM)-treated (E) cortical cultured neurons. FIG. 1F, FIG. 1G, FIG. 1H, FIG. 1i, FIG. 1J, FIG. 1K, FIG. 1L, FIG. 1M: Immunoblots and corresponding quantifications showing time-dependent increases in the level of carbonylated proteins (FIG. 1F, FIG. 1J), increased levels of phosphorylated extracellular-signal related kinase 1/2 (ERK1/2) and glycogen synthase kinase (GSK-3β) (FIG. 1G, FIG. 1K), enhanced levels of phosphorylated tau, cleaved-tau and cleaved-caspase-3 (FIG. 1H, FIG. 1L) and enhanced levels of autophagic markers LC3-II and ATG5, decreased levels of beclin-1 and unaltered P62 levels (I, M) in total cell lysate of neurons treated with 10 μM Aβ₁₋₄₂. FIG. 1N & FIG. 1O: LysoTracker DND-99 labelling of endosomes/lysosomes revealed punctate staining in control (N) but diffuse staining in 24 h Aβ₁₋₄₂ (10 μM)-treated (O) cultured neurons. All cells were labelled with Hoechst 33258. FIG. 1P & FIG. 1Q: LysoSensor DND-160 labelling of control (FIG. 1P) and 24 h Aβ₁₋₄₂ (10 μM)-treated (FIG. 1Q) cultured neurons. All blots were re-probed with anti actin. All data expressed as mean±SEM were obtained from three to five separate experiments. CTL, control. *p<0.05, **p<0.01, ***p<0.001. Scale bar=10 μm or 50 μm.

FIG. 2. Native PLGA nanoparticles protect neurons against Aβ₁₋₄₂-induced toxicity A: Immunofluorescence images of cultured neurons showing lysosomal labelling with anti-LAMP1 antibody (FIG. 2A), fluorescence PLGA labelling following internalization (FIG. 2A′) and their colocalization (FIG. 2A″). Note the colocalization of PLGA on LAMP1-labelled lysosomes in cultured neurons. FIG. 2B FIG. 2C, FIG. 2D: Histograms depicting protective effects of native PLGA against 10 μM Aβ₁₋₄₂-mediated toxicity following pre-treatment (FIG. 2B), co-treatment (FIG. 2C) and post-treatment (FIG. 2D) paradigms as revealed by MTT assay. Note that native PLGA can differentially protect the cultured neurons against Aβ-mediated toxicity depending on the experimental paradigm. All results, which are presented as means±SEM, were obtained from three to five separate experiments. **p<0.01, ***p<0.001. Scale bar=10 μm.

FIG. 3. Native PLGA attenuates Aβ₁₋₄₂-induced lysosomal abnormalities and signaling mechanism FIG. 3A, FIG. 3B, FIG. 3C, FIG. 3D: LysoTracker DND-99 labelling in control (FIG. 3A), 10 μM Aβ₁₋₄₂ (FIG. 3B), 200 μg/ml PLGA (FIG. 3C) and 10 μM Aβ₁₋₄₂+200 μg/ml PLGA (FIG. 3D) treated cultured neurons. Note that PLGA treatment partially reversed diffuse LysoTracker DND-99 staining of Aβ₁₋₄₂-treated cultured neurons. All cells were labelled with Hoechst 33258. E-H: LysoSensor DND-160 labelling in control (FIG. 3E), 10 μM Aβ₁₋₄₂ (FIG. 3F), 200 μg/ml PLGA (FIG. 3G) and 10 μM Aβ₁₋₄₂+200 μg/ml PLGA (FIG. 3H) treated cultured neurons. Note that PLGA treatment partially reversed basic pH environment observed in Aβ₁₋₄₂-treated cultured neurons. FIG. 3I, FIG. 3J, FIG. 3K, FIG. 3L, FIG. 3M, FIG. 3N: Immunoblots (FIG. 3I, FIG. 3J, FIG. 3K) and corresponding quantifications (FIG. 3L, FIG. 3M, FIG. 3N) showing that 50 μM PLGA treatment attenuated Aβ₁₋₄₂-induced increased levels of phosphorylated extracellular-signal related kinase 1/2 (ERK1/2) and glycogen synthase kinase (GSK-3β) (FIG. 3I, FIG. 3L), enhanced levels of phosphorylated tau, cleaved tau and cleaved-caspase-3 (FIG. 3J, FIG. 3M) and increased levels of carbonylated proteins (FIG. 3K, FIG. 3N) in mouse cortical cultured neurons. All results, which are presented as means±SEM, were obtained from three to five separate experiments. CTL, control. *p<0.05, **p<0.01, ***p<0.001. Scale bar=10 μm.

FIG. 4. Spontaneous aggregation of Aβ₁₋₄₂ A: Increased Thioflavin T signal with increasing concentrations (1-20 μM) of Aβ₁₋₄₂. FIG. 4B: Thioflavin T-stained fluorescence images of Aβ₁₋₄₂ fibers with increasing concentrations (1-20 μM) of the peptide. FIG. 4C: Transmission electron micrographs depicting the presence of mature Aβ fibers after aggregation. FIG. 4D & FIG. 4E: DLS data revealing hydrodynamic radius of monomeric and aggregated Aβ₁₋₄₂. FIG. 4F: Zeta potential measurement for Aβ₁₋₄₂ displaying surface charge of −32 mV. FIG. 4G: Native PAGE of Aβ₁₋₄₂ aggregates showing the presence of higher ordered fibrillar entities. FIG. 4H: CD spectra confirming the formation of beta sheet following aggregation of Aβ₁₋₄₂. I: FTIR showing structural conformation Aβ₁₋₄₂ after aggregation.

FIG. 5. Attenuation of spontaneous Aβ₁₋₄₂ aggregation by PLGA A: Graphical representation showing the polymerization of poly L-lactic acid and poly L-glycolic acid to form PLGA nanoparticles. FIG. 5B: Transmission electron micrographs showing the spheroidal nature of PLGA nanoparticles with an average diameter of ˜100 nm. FIG. 5C: DLS histogram of the PLGA nanoparticles displaying the diameter size of ˜100 nm. FIG. 5D: Zeta potential measurement for PLGA nanoparticles revealing the surface charge of −8 mV. E: PLGA dose-dependently (2.5-50 μM) attenuate spontaneous aggregation of 10 μM Aβ₁₋₄₂ as revealed by Thioflavin T fluorescence assay. FIG. 5F: Thioflavin T-stained fluorescence images depicting attenuation of PLGA-induced Aβ₁₋₄₂ aggregation. FIG. 5G: Transmission electron micrographs showing direct association of PLGA nanoparticles with Aβ₁₋₄₂ fibers.

FIG. 5H & FIG. 5I: DLS histograms revealing hydrodynamic radius of 10 μM Aβ₁₋₄₂ in the absence (FIG. 5H) and presence of 50 μM PLGA (FIG. 5I). Note the decrease in the hydrodynamic radius of Aβ₁₋₄₂ fibers in the presence of PLGA species. FIG. 5J: Native PAGE showing the loss of higher ordered entities in presence of PLGA nanoparticles and the corresponding dot blot revealing decreased formation of Aβ₁₋₄₂ fibers in presence of PLGA as detected by a fibril specific OC antibody. FIG. 5K: FTIR secondary derivative spectra of the aggregating Aβ₁₋₄₂ samples in presence and absence of PLGA nanoparticles. FIG. 5L: CD spectra showing decreased beta sheet formation following incubation of 10 μM Aβ₁₋₄₂ in the presence of 50 μM PLGA nanoparticles.

FIG. 6. Specificity and molecular interactions of Aβ₁₋₄₂ with PLGA FIG. 6A: Thioflavin T kinetic graph showing that PLGA with 50:50 resomer (red color) from a different source was able to suppress spontaneous Aβ₁₋₄₂ aggregation, but PLGA with 75:25 resomer (purple color) did not affect Aβ aggregation. FIG. 6B: Thioflavin T kinetic graph showing that aggregation of 10 μM Aβ₁₋₄₂ was not altered by the presence of 50 μM lactic acid, 50 μM glycolic acid or a mixture of 50 μM lactic acid+glycolic acid. FIG. 6C: Isothermal titration calorimetry data obtained after base line subtraction for the titration of 600 μM PLGA with 10 μM Aβ₁₋₄₂ at 37° C. The data were plotted for a single site binding model and the thermodynamic parameters for the protein and ligand complex interaction are presented in the inset. FIG. 6D: Decreased fluorescence emission of fluorescent PLGA nanoparticles in the presence of Aβ₁₋₄₂ monomers revealing the quenching effect during interaction. FIG. 6E: Molecular docking data using Auto dock Vina revealing the interaction of PLGA with the monomeric Aβ₁₋₄₂ PDB ID:1IYT. FIG. 6F: Epitope mapping using dot blot revealing that PLGA nanoparticles interact with the hydrophobic domain of Aβ₁₋₄₂.

FIG. 7. Specificity of PLGA effects on aggregation of different isoforms of Aβ peptides FIG. 7A, FIG. 7B, FIG. 7C, FIG. 7D, FIG. 7E, FIG. 7F, FIG. 7G, FIG. 7H, FIG. 7I, FIG. 7J, FIG. 7K, FIG. 7L, FIG. 7M, FIG. 7N, FIG. 7O: Thioflavin T aggregation kinetics and respective fluorescence images showing the effects of 50 μM PLGA on the aggregation of 100 μM Aβ₁₋₄₀ (FIG. 7A, FIG. 7B, FIG. 7C), 400 μM Aβ₁₇₋₄₂ (FIG. 7D, FIG. 7E, FIG. 7F), 400 μM Aβ₂₅₋₃₅ (FIG. 7G, FIG. 7H, FIG. 7I), 10 μM Iowa mutant Aβ₁₋₄₂ (FIG. 7J, FIG. 7K, FIG. 7L) and 10 μM Aβ₁₋₄₂ (FIG. 7M, FIG. 7N, FIG. 7O). Control Aβ₁₋₄₂, as expected, did not exhibit significant aggregation, thus validating the specificity of aggregation kinetic assay. Note that PLGA, as observed for Aβ₁₋₄₂, is able to attenuate the spontaneous aggregation of various other Aβ fragments.

FIG. 8. Disassembly of aggregated Aβ₁₋₄₂ fibers by PLGA A & B: PLGA dose-dependently (2.5-50 μM) triggered disassembly of aggregated Aβ₁₋₄₂ as revealed by the Thioflavin T fluorescence assay (FIG. 8A) and Thioflavin T stained fluorescence images (FIG. 8B). FIG. 8C: Thioflavin T stained images of mature Aβ₁₋₄₂ fibers before and after treatment with 50 μM PLGA revealing the untwining effect after disassembly reaction. FIG. 8 D: Fluorescence imaging of Congo red stained Aβ₁₋₄₂ fibers in the presence of green fluorescent PLGA showing direct interaction between Aβ₁₋₄₂ fibers and PLGA. FIG. 8E: Transmission electron micrographs showing matured Aβ₁₋₄₂ fibers in the absence and presence of 50 μM PLGA depicting the direct interaction of the nanoparticles with Aβ fibers.

FIG. 8F, FIG. 8G, FIG. 8H, FIG. 8I: DLS histograms revealing hydrodynamic radius of aggregated Aβ₁₋₄₂ in the absence (FIG. 8F) and presence of different concentrations of PLGA (FIG. 8G, FIG. 8H, FIG. 8I). Note the shift in the hydrodynamic radius of Aβ₁₋₄₂ fibers towards the lower ordered species and possible release of monomers in the presence of PLGA nanoparticles. FIG. 8J: Native PAGE showing the loss of higher ordered Aβ₁₋₄₂ entities in presence of PLGA. FIG. 8K: Fluorescence quenching effect of labelled PLGA in the presence of increasing concentrations of matured Aβ₁₋₄₂ fibers displaying a decrease in fluorescence intensity, suggesting an interaction between PLGA and Aβ₁₋₄₂ fibers.

FIG. 9. PLGA-mediated attenuation of Aβ₁₋₄₂ aggregation protected neurons against toxicity A & B: Histograms showing protection of mouse cortical cultured neurons following attenuation of spontaneous aggregation of 10 μM Aβ₁₋₄₂ by 25 and 50 μM PLGA as detected with MTT (FIG. 9A) and LDH (FIG. 9B) assays. C-J: Immunoblots and corresponding histograms showing that protective effects following spontaneous Aβ aggregation by PLGA is mediated by decreasing the levels of Phospho-Tyr GSK-3β (FIG. 9C, FIG. 9D), Phospho-ERK1/2 (FIG. 9E, FIG. 9F), Phospho-tau (FIG. 9G, FIG. 9H) and cleaved-caspase-3 (FIG. 9I, FIG. 9J) levels induced by Aβ peptide. FIG. 9K & FIG. 9L: Histograms showing protection of mouse cortical cultured neurons following disassembly of Aβ₁₋₄₂ aggregates with different concentrations of PLGA as detected with MTT (FIG. 9K) and LDH (FIG. 9L) assays.

FIG. 10. PLGA-mediated attenuation of AD-related cognitive deficit and pathology in 5×FAD mice FIG. 10A: Photomicrographs depicting OC antibody labelled Aβ deposits in the cortex (FIG. 10A i-iv) and cerebellum (FIG. 10A v-iii) of CSF- and PLGA-treated WT and 5×FAD mouse brain tissues. As expected, Aβ deposits are evident in the cortex but not in the cerebellum of 5×FAD mouse brains. WT mouse did not depict Aβ deposits in any brain regions. FIG. 10B: Histograms showing the quantification of OC antibody labelled Aβ plaques number (FIG. 10B i), size (FIG. 10B ii) and areas occupied (FIG. 10B iii) in the cortical (i.e., frontal, parietal and entorhinal) regions of CSF- and PLGA-treated 5×FAD mice. Note the decreased number/size and areas occupied by Aβ-containing neuritic plaques in PLGA-treated 5×FAD mice compared to CSF-treated 5×FAD mice. FIG. 10C and FIG. 10D: Immunoblots (FIG. 10C; FIG. 10C i and FIG. 10Cii) and their quantification (FIG. 10D; FIG. 10D i-iii) showing the levels of APP holoprotein (Y188) and APP-CTFs (i.e., αCTF and βCTF) in the parietal cortex (FIG. 10C; FIG. 10Ci) and cerebellum (FIG. 10C; FIG. 10Cii) of CSF- and PLGA-treated 5×FAD and WT mouse brain tissues. Note the decreased levels of APP and its cleaved products in the cortex of PLGA-treated 5×FAD mice compared to CSF-treated 5×FAD mice. The quantifications of the respective blots were represented as a percentage of the corresponding control group (FIG. 10D; FIG. 10D i-iii). FIG. 10E: Histograms depicting decreased cortical levels of Aβ₁₋₄₀ (FIG. 10Di) and Aβ₁₋₄₂ (FIG. 10Dii) in PLGA-treated 5×FAD mice compared to CSF-treated 5×FAD mice. FIG. 10F: Histograms showing the reversal of object recognition memory following PLGA treatment in 5×FAD mice. The data represent the total number of visits to novel object (i) and the discrimination index (ii) from familiar to the novel object of PLGA- and CSF-treated animals. All data expressed as mean±SEM were obtained from three replicates. CSF, artificial cerebrospinal fluid; WT, wildtype. *p<0.05, **p<0.01, ***p<0.001.

FIG. 11. FIG. 11A, FIG. 11B, FIG. 11C, FIG. 11D: Histograms depicting dose-(FIG. 11A, FIG. 11C) and time-(FIG. 11B, FIG. 11D) effects of native PLGA on the viability of mouse cortical cultured neurons as evaluated by MTT (FIG. 11A, FIG. 11B) and LDH (FIG. 110, FIG. 11D) assays. Note that 25-200 μg/ml native PLGA did not affect neuronal viability over 24 h time period, whereas viability was affected when neurons were treated for 24 h with 500 μg/ml. Additionally, 100 μg/ml PLGA over 48 h period did not affect cell viability as evident by MTT and LDH assays. All data expressed as mean±SEM were obtained from three to five separate experiments. CTL, control. **p<0.01, ***p<0.001.

FIG. 12A & FIG. 12B: Stability of 50 μM PLGA nanoparticles at 0 h (FIG. 12A) and 240 h (FIG. 12B) as detected by DLS.

FIG. 13A, FIG. 13B, FIG. 13C, FIG. 13D, FIG. 13E, FIG. 13F, FIG. 13G, FIG. 13H: Thioflavin T aggregation kinetics and respective fluorescence images showing the effects of 25 μM PLGA on the aggregation of 1 μM Aβ₁₋₄₂ (FIG. 13A, FIG. 13B), 5 μM Aβ₁₋₄₂ (FIG. 13C, FIG. 13D), 10 μM Aβ₁₋₄₂ (FIG. 13E, FIG. 13F) and 20 μM Aβ₁₋₄₂ (FIG. 13G, FIG. 13H). Note that 25 μM PLGA can able to attenuate Aβ₁₋₄₂ aggregation at all studied concentrations as a function of time.

FIG. 14A, FIG. 14B, FIG. 14C, FIG. 14D, FIG. 14E, FIG. 14F, FIG. 14G, FIG. 14H: Thioflavin T aggregation kinetics and respective fluorescence images showing the effects of 50 μM PLGA on the aggregation of 1 μM Aβ₁₋₄₂ (FIG. 14A, FIG. 14B), 5 μM Aβ₁₋₄₂ (FIG. 14C, FIG. 14D), 10 μM Aβ₁₋₄₂ (FIG. 14E, FIG. 14F) and 20 μM Aβ₁₋₄₂ (FIG. 14G, FIG. 14H). Note that 50 μM PLGA can able to attenuate Aβ₁₋₄₂ aggregation at all studied concentrations as a function of time.

FIG. 15. Fluorescence quenching of Aβ₁₋₄₂ monomers with florescence tagged PLGA nanoparticles. FIG. 15A: Stern Volmer plot for PLGA and Aβ₁₋₄₂ predicted as Ksv=1.91×10⁵ M⁻¹. FIG. 15B: The binding plot for PLGA with Aβ₁₋₄₂ log [(F0−F)/F] versus log [Q] displaying a single binding site with a Kd value of 9.09×10⁴M.

FIG. 16. Fluorescence quenching of Aβ₁₋₄₂ fibers with florescence tagged PLGA nanoparticles. FIG. 16A: Stern Volmer plot for PLGA and Aβ₁₋₄₂ fibers predicted a Ksv=1×10⁶M⁻¹. FIG. 16B: The binding plot for PLGA with Aβ₁₋₄₂ log [(F0−F)/F] versus log [Q] displaying a single binding site with a Kd value of 6.95×10⁷M.

FIG. 17A, FIG. 17B, FIG. 17C. Docked complex of monomeric Aβ₁₋₄₂ (PDB ID: 1IYT) with PLGA. The interaction energy parameter=−4.4 kcal·mol⁻¹.

FIG. 18A, FIG. 18B. Docked complex monomeric Aβ₁₋₄₂ (PDB ID: 5OQV) with PLGA. The interaction energy parameter=−4.3 kcal·mol⁻¹.

FIG. 19. Thioflavin T fluorescence images showing the disassembly of 10 μM Aβ₁₋₄₂ aggregates in the presence or absence of 2.5, 25 and 50 μM PLGA as a function of time.

FIG. 20A & FIG. 20B; Histograms showing protection of mouse cortical cultured neurons following disassembly of 10 μM Aβ₁₋₄₂ aggregates over 120 h duration with different concentrations of PLGA as detected with MTT (FIG. 20A) and LDH (FIG. 20B) assays.

### DETAILED DESCRIPTION

In one aspect, there is described a method of treating a subject having Alzheimer's disease (AD) or suspected of having Alzheimer's disease (AD), comprising administering to a subject in need thereof a therapeutically effective amount of poly(D,L-lactide-co-glycolide) (PLGA).

In one aspect there is described a use of a therapeutically effective amount of poly(D,L-lactide-co-glycolide) (PLGA) for the treatment of AD in a subject. Such therapeutically effective amount of PLGA polymer may comprise a ratio of lactic to glycolic acid of about 65:35, 60:40, 55:45, 50:50, 45:55, 40:60, or 35:65.

In one aspect there is described a use of a therapeutically effective amount of poly(D,L-lactide-co-glycolide) (PLGA), in the manufacture of a medicament for the treatment of AD in a subject.

In one aspect, there is described a method of treating a subject having a tauopathy or suspected of having a tauopathy, comprising administering to a subject in need thereof a therapeutically effective amount of poly(D,L-lactide-co-glycolide) (PLGA).

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

The term “Alzheimer's Disease” (AD) generally refers to a mental deterioration in a subject, where clinical manifestations may include, but are not limited to, progressive memory deficits, confusion, behavioral problems, inability to care for oneself, gradual physical deterioration and, ultimately, death.

As used herein, Alzheimer's Disease may include preclinical Alzheimer's dementia, Mild cognitive impairment, and/or Alzheimer's.

As used herein, “tauopathy” generally refers to a neurodegenerative disease involving an aggregation of tau protein in the brain. Tauopathies may include, but are not limited to, frontotemporal dementia, Pick's disease, epilepsy, and chronic traumatic encephalopathy.

The term “therapeutically effective amount”, as used herein, refers to an amount effective, at dosages and for periods of time necessary to achieve the desired result. Effective amounts may vary according to factors such as the disease state, age, sex and/or weight of the subject. The amount of a given compound or composition that will correspond to such an amount will vary depending upon various factors, such as the given drug or compound, the pharmaceutical formulation, the route of administration, the identity of the subject being treated, and the like, but may be determined by one skilled in the art.

The term “treatment” or “treat” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject in the early stage of disease can be treated to prevent progression or alternatively a subject in remission can be treated with a compound or composition described herein to prevent progression.

In some examples, treatment methods and/or uses comprise administering to a subject a therapeutically effective amount of a compound or composition described herein and optionally consists of a single administration or application, or alternatively comprises a series of administrations or applications.

In some examples, administration may be accomplished in vitro, in vivo, or ex vivo.

In one embodiment, the present invention encompasses administering the compounds of the present invention to a subject.

Administration may be in vitro, ex vivo or in vivo.

Administering can also be performed, for example, once, a plurality of times, and/or over one or more extended periods.

Administration may be by any suitable means.

The compounds herein may be formulated with pharmaceutically acceptable carriers, excipients or diluents.

Pharmaceutically acceptable carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Compositions as described herein may be sterilized by conventional methods and/or lyophilized.

Routes of administration of the compounds and/or compositions described herein include, but are not limited to, intracerebroventricular, oral (e.g., by ingestion); buccal; sublingual; transdermal (including, e.g., by a patch, plaster, etc.); transmucosal (including, e.g., by a patch, plaster, etc.); intranasal (e.g., by nasal spray, drops or from an atomizer or dry powder delivery device); ocular (e.g., by eye drops); pulmonary (e.g., by inhalation or insufflation therapy using, e.g., an aerosol or vapour, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

In some examples, the other or additional treatment further comprises administering a therapeutically effective amount of an existing treatment of Alzheimer's Disease.

Methods of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in any way.

### EXAMPLES

### Summary

Evidence suggests that increased level/aggregation of beta-amyloid (Aβ) peptides, generated from amyloid precursor protein (APP), initiate neurodegeneration and subsequent development of Alzheimer's disease (AD)—the prevalent cause of dementia affecting elderly. At present, there is no effective treatment for AD. A number of studies, however, have focused on developing small molecules, including conjugated nanoparticles that can prevent Aβ toxicity and/or aggregation as a treatment strategy for AD, but little is known about the effects of unconjugated nanoparticles such as poly(D,L-lactide-co-glycolide) (PLGA)—which is used as a carrier to deliver drugs into the central nervous system. Since Aβ toxicity is mediated, at least in part, by breakdown of lysosomal membrane permeability that can be restored by PLGA, we evaluated the effects of unconjugated PLGA on Aβ aggregation as well as in cellular and animal models of AD—as a potential treatment strategy for AD pathology. Our results showed that Aβ₁₋₄₂-induced toxicity in mouse primary cortical neurons is associated with an impaired lysosomal integrity, enhanced levels of carbonylated proteins and phosphorylation of tau protein. Interestingly, the treatment of neurons with unconjugated PLGA attenuated Aβ toxicity by restoring lysosomal leakage and reducing the levels of carbonylated proteins as well as tau phosphorylation. In parallel, we showed that PLGA can suppress the spontaneous aggregation of various isoforms of Aβ peptides. Our biophysical, structural and epitope mapping assays revealed a strong interaction of PLGA with the aggregation-prone hydrophobic domain of Aβ peptides. Additionally, we showed that PLGA can trigger disassembly of preformed Aβ fibers—a feature relevant to AD pathology. Our data further indicated that Aβ samples collected following PLGA treatment significantly enhanced neuronal viability compared to PLGA untreated Aβ samples by reducing the levels of carbonylated proteins and phosphorylation of tau protein. Finally, we showed that intracerebroventricular administration of PLGA for 28 days using osmotic pump into 3 month old 5×FAD animal model of AD can significantly attenuate object recognition memory deficit and Aβ-containing neuritic plaques in the affected cortical regions of the brain. Collectively, these results suggest that PLGA without conjugation with any drug/agent i) can protect neurons against Aβ-mediated toxicity by restoring lysosomal leakage and impaired cellular mechanisms, ii) can suppress not only the spontaneous aggregation but also can trigger disassembly of aggregated Aβ, both of which can protect neurons against Aβ toxicity and iii) can reverse cognitive deficits and AD-related pathology in 5×FAD mouse model of AD—thus signifying the unique therapeutic potential of PLGA in the treatment AD pathology.

Material and Methods

Materials: Dulbecco's modified Eagle's medium (DMEM), neurobasal medium, Hanks' balanced salt solution (HBSS), fetal bovine serum (FBS), B27, N2 supplement, Alexa Fluor 488/594 conjugated secondary antibodies, ProLong Gold anti-fade reagent and ELISA kits for the detection of mouse/human Aβ₁₋₄₀ and Aβ₁₋₄₂ were purchased from R&D systems (Minneapolis, Minn., USA). The bicinchoninic acid (BCA) protein assay kit and enhanced chemiluminescence (ECL) kit were from Thermo Fisher Scientific Inc. (Nepean, ON, Canada), whereas LysoSensor Yellow/Blue DND-160 was from Invitrogen (Eugene, Oreg., USA). The ALZET® mini-osmotic pumps (model 2004) and brain infusion kits were procured from DURECT Corporation (Cupertino, Calif., USA). Isoforms of Aβ₁₋₄₂, Aβ₁₋₄₀, Aβ₁₇₋₄₂, Aβ₂₅₋₃₅ and the reverse sequence of Aβ₁₋₄₂ (i.e. Aβ₁₋₄₂) were purchased from R Peptide (Sunnyvale, Calif., USA), whereas D23N Iowa mutant Aβ₁₋₄₂ was procured from Ana Spec (Sunnyvale, Calif., USA). Degradex® PLGA (50:50 resomer) and fluorescent Degradex® PLGA were obtained from Phosphorex (Hopkinton, Mass., USA). Thioflavin T (ThT), hexafluoro-2-propanol (HFIP), PLGA (50:50 and 75:25 resomers) and 3-(4,5-dimethylthiazol-2-yl)-2,5-diphenyltetrazolium bromide (MTT) were obtained from Sigma-Aldrich (St. Louis, Mo., USA), the lactate dehydrogenase (LDH)-based cytotoxicity assay kit was purchased from Promega (Madison, Wis., USA) and the Oxyblot™ protein oxidation kit was from EMD Millipore (Burlington, Mass., USA). Electron microscopy grids (Formvar/Carbon 300 mesh, Copper with grid hole size 63 μm) and uranyl acetate stain were purchased from TedPella (Redding, Calif., USA). Sources of primary antibodies used in the study are listed in Table-1. The horseradish peroxidase-conjugated secondary antibodies were purchased from Santa Cruz Biotechnology (Paso Robles, Calif., USA). All other chemicals were obtained from either Sigma-Aldrich or Thermo Fisher Scientific.

Preparation of Aβ peptides: All lyophilized Aβ₁₋₄₂, Aβ₁₋₄₀, Aβ₁₇₋₄₂, Aβ₂₅₋₃₅, Aβ₄₂₋₁ and D23N Iowa mutant Aβ₁₋₄₂ isoforms stored at −80° C. were first equilibrated at room temperature for 30 min prior to dissolving in HFIP to obtain a 1 mM solution. Once dissolved, peptide aliquots were quickly dried down using a SpeedVac to remove HFIP and then restored at −80° C. for subsequent use as described earlier (60). Just prior to the experiments, various isoforms of Aβ peptides were thawed at 4° C., diluted first with dimethyl sulfoxide (DMSO) to 5 mM concentration and then to 100 μM concentrations with sterile dH₂O. For the preparation of Aβ fibrils, diluted peptides were incubated at 37° C. overnight in phosphate-buffered saline (PBS, pH 7.4), whereas for the oligomer formation, peptides were incubated at 4° C. in PBS overnight.

Mouse cortical neuronal cultures and treatments: Timed pregnant BALB/c mice purchased from Charles River (St. Constant, QC, Canada) were maintained according to Institutional and the Canadian Council on Animal Care guidelines. Primary cortical cultures were prepared from 18-day-old embryos of timed pregnant mice as described previously (60, 61). In brief, frontal cortex from pup brains was dissected in Hanks' balanced salt solution supplemented with 15 mM HEPES, 10 units/mL penicillin, and 10 μg/ml streptomycin and then digested with 0.25% trypsin-EDTA. The cell suspension was filtered through a cell strainer and plated (5×10⁴ cells/cm²) on either 96-well plates (for survival/death assay), 6-well plates (1.5×10⁶ cells/cm² for biochemical analysis) or 8-well chamber slides (1×10⁵ cells/cm² for LysoSensor DND-160 labelling and immunocytochemistry). The cultures were grown at 37° C. in a 5% CO₂ humidified atmosphere in Neurobasal medium supplemented with B27/N2, 50 μM L-glutamine, 15 mM HEPES, 10 units/ml penicillin, 300 μg/ml streptomycin and 1% FBS. The medium was replaced 1 day later without FBS and neurons were treated on day 6 or 7 after plating as described previously (60, 61). In brief, cultured neurons were treated with oligomeric 1-20 μM Aβ₁₋₄₂ for 24 h or with oligomeric 10 μM Aβ₁₋₄₂ for different periods of times (6-48 h). In some experiments, neurons were treated with only different concentrations of PLGA (25-500 μg/ml) for 24 h or 100 μg/ml PLGA for various times (i.e., 6-48 h). In parallel, cultured neurons were pre-treated with PLGA (100 or 200 μg/ml) for 24 h followed by exposure to 10 μM Aβ₁₋₄₂ for an additional 24 h or neurons were co-treated with PLGA (100 or 200 μg/ml) along with 10 μM Aβ₁₋₄₂ for 24 h or 48 h. In a subsequent set of experiments, neurons were post-treated with PLGA (100 or 200 μg/ml) for either 12 h or 24 h following exposure to 10 μM Aβ₁₋₄₂ for 12 h or 24 h. In a parallel set of experiment, cultured neurons were treated with aggregated Aβ₁₋₄₂ either alone or with aggregated Aβ₁₋₄₂ incubated with 25 or 50 μM PLGA for either 24 or 120 h. Control and Aβ-treated cultures from various experimental paradigms were then processed for cell toxicity/viability assays, LysoSensor Yellow/Blue DND-160 labelling, Western blotting and/or immunostaining.

Cytotoxicity assays: Neuronal viability following various experimental paradigms was analysed using MTT and LDH assays as described earlier (60-63). For the MTT assay, control and Aβ-treated culture plates were replaced with new media containing 0.5 mg/ml MTT and then incubated for 4 h at 37° C. in 5% CO₂/95% air. The formazan was dissolved in dimethyl sulfoxide and absorbance was measured at 570 nm with a microplate reader. To substantiate MTT data, control and Aβ-treated cultured neurons were processed for a cytotoxicity assay based on the measurement of LDH activity released into the conditioned medium from the cytosol of damaged cells (60-63). The absorbance was measured at 490 nm with a Spectramax M5 spectrophotometer. Both MTT and LDH experiments were repeated three to five times with three technical replicates per sample.

ThT fluorescence assay: A universally used ThT fluorescent dye is utilized for detecting the amyloid fibril formation (60, 64, 65). The aggregation kinetics of different concentrations (1-20 μM) or isoforms of Aβ peptides were carried out at mimicked physiological conditions in 100 μL reaction buffer (10 mM Na₂HPO₄ with 40 mM NaCl; pH 7.4) at 37° C. for 24 h in the presence and absence of various concentrations of PLGA (5-50 μM). The concentration of ThT was maintained at 30 μM throughout the experiment (60). For Aβ disassembly experiments ThT signal was monitored for the mature aggregates in the presence of PLGA at regular time intervals. The fluorescence was measured every 15 min over 48-120 h with an excitation at 440 nm and emission at 480 nm with a cut off filter at 475 nm using a FLUOstar omega BMG Labtech (Aylesbury, UK) or a Spectra max M5 (Molecular Devices, San Jose, Calif., USA). All kinetics experiments were repeated nine times with three technical replicates for each sample/experiment and the data are presented as mean±SEM. The kinetic traces of experiments were normalized and represented in the form of relative fluorescence intensity and the graphs were plotted using ORIGIN 2020 software.

Fluorescence Microscopy: After incubation of various Aβ samples with or without PLGA for different periods of time (0-120 h), 100 μl samples were transferred to glass slides, stained with ThT, dried at 37° C. and then examined using a Nikon 90i fluorescence microscopic as described previously (66, 67). All photomicrographs of ThT-labelled samples were captured at 20× magnification.

Transmission Electron microscopy (TEM): PLGA alone and Aβ₁₋₄₂ aggregates in the presence or absence of PLGA were placed on 200 mesh carbon-coated copper grids for ˜2 min followed by a series of washing with ammonium acetate buffer. The washed samples were negatively stained by 2% Na phosphotungstic acid for 10 min, washed, dried and then examined under accelerating voltage of 200 kV using TEM. The negatively stained samples were analysed with a FEI Tecnai G20 TEM and micrographs were recorded using an Eagle 4 k×4 k CCD camera (FEI Company).

Dynamic Light scattering (DLS): The hydrodynamic radii of PLGA alone and Aβ₁₋₄₂ aggregates with or without PLGA were measured using a Malvern Zetasizer Nano ZS (Malvern Instruments, MA, USA) equipped with a back-scattering detector (173 degrees). The nanoparticle samples were prepared by filtering them through a pre-rinsed 0.2-μm filter and all readings were recorded after equilibrating samples for 5 min at 25° C. Particle size was calculated by the manufacturer's software through the Stokes-Einstein equation assuming spherical shapes of the particles.

Circular dichroism (CD) spectroscopy: The changes in the secondary structure of aggregated Aβ were determined using a Chirascan qCD spectropolarimeter as described earlier (67). After incubating 10 μM Aβ₁₋₄₂ in the presence or absence of 50 μM PLGA, the reaction mixture was subjected to spectroscopic studies and CD spectra were recorded at room temperature in a CD cuvette of 2 mm path length. The reported spectra were the average of 9 different acquisitions between 200 and 260 nm. The alteration in the secondary structure of Aβ₁₋₄₂ was determined by analyzing the ellipticity values of the samples taken from the aggregation reactions with or without PLGA using online server BeSTSel.

Isothermal Titration calorimetry (ITC): The thermodynamics of Aβ₁₋₄₂ and PLGA were studied using a MicroCal ITC at 298K in a high feedback gain mode with a differential power of 10 μcal/sec at 250 rpm as described earlier (68, 69). In brief, Aβ₁₋₄₂ (10 μM) was injected with a flow rate of 0.25 μl/min into to the sample cell containing 600 μM PLGA nanoparticles. The reference cell was filled with degassed water. All samples were degassed at 4° C. before titration to prevent the possible formation of air bubbles in the sample cell. To correct for the heat of dilution, standard experiments were performed for PLGA with PBS, PBS alone, Aβ₁₋₄₂ in the presence or absence of PLGA. The obtained data were subtracted from the respective sample titrations. All data were analyzed and fitted for a single site of interaction with the inbuilt ORIGIN 2020 software.

Molecular docking: The molecular interaction between the Aβ peptide and PLGA was studied by using AutoDock Vina wizard of PyRx(v0.8) (70). The structures of the Aβ₁₋₄₂ monomer (PDB ID 1IYT) and Aβ₁₋₄₂ fibers (5OQV, 2BEG) were obtained from RSCB (Protein Data Bank), whereas the three-dimensional structure of PLGA was constructed using Molden and Discovery studio visualizer 2019. All protein molecules were pre-processed and protonated prior to conducting docking experiments using an Auto Dock Vina platform in which each protein molecule was docked with the PLGA in a manually defined grid box. After docking, the results were screened on the basis of energy values and the selected pose cluster was visualized using Discovery studio visualizer 2019 and Chimera 14.1 as described earlier (71).

Fluorescence Quenching: The interaction between the protein and ligand was quantified by using fluorescence quenching experiment as described previously (72, 73). All samples were prepared in PBS and the interaction between Aβ₁₋₄₂ monomers, Aβ₁₋₄₂ fibers and fluorescent labelled PLGA was recorded using a Spectra Max M5. The concentration of PLGA was kept constant at 2 μM, whereas the concentration of Aβ₁₋₄₂ was varied between 42 to 294 nM. On the contrary, for the quenching studies for Aβ₁₋₄₂ fibers with PLGA the concentration of 10 μM PLGA was selected with the varied concentrations of Aβ₁₋₄₂ fibers from 42 to 294 nM. The PLGA was excited at 440 nm, and the slit width was fixed to 10 nm for excitation and emission. The emission spectra were collected from 450 to 650 nm at room temperature. Binding constants were calculated using fluorescence intensity values at the maximum emission of 500 nm. From these values we calculated the Stern Volmer constant, predicted the nature of binding constant and the number of binding sites. The obtained fluorescence spectra for the PLGA were analyzed by using the Stern Volmer equation (74).

F₀/F=1+τ₀k_{q}[Q]=1+Kₛᵥ[Q]

F₀=Fluorescence intensity in the absence of quencher, F=Fluorescence intensity in the presence of quencher, Q=Concentration of the quencher.

The data were plotted and the slope value of the linear fit Stern Volmer constant [Kₛᵥ] was determined. From this we can predict if binding of PLGA with the Aβ₁₋₄₂ is static or dynamic using the formula Kₛᵥ=τₒ[k_{q}] where τₒ is the average life-time of any biomolecule. The binding site (n) and the binding constant (Ka) of Aβ₁₋₄₂ to PLGA was calculated by the formula:

$$log \frac{Fo - F}{F} = log ( Ka ) + n log [ Q ] \vert$$

F₀=Fluorescence intensity in the absence of quencher, F=Fluorescence intensity in the presence of quencher, Q=Concentration of the quencher, Ka=binding constant, n=number of binding sites.

The values of log [(F₀−F)/F] were calculated and a graph was plotted against the calculated log [Q] values. A linear fit of the data was carried out to extract important parameters such as binding constant (Ka) and number of binding sites (n).

Fourier-transform infrared (FTIR) spectroscopy: FTIR spectroscopy study was performed to identify the secondary structural vibration of the aggregated Aβ₁₋₄₂ in the presence or absence of 50 μM PLGA. ATR mode was used to obtain the secondary derivative (1700 cm⁻¹ and 1600 cm⁻¹) spectra. All the spectra were taken from a Bruker Vertex 70 spectrometer equipped with a silicon carbide source and MCT detector. The spectral readings were processed using OPUS 6.5 software (66, 75, 76).

Native polyacrylamide gel electrophoresis (PAGE) analysis: We used Native PAGE to determine the higher order entities of the aggregated Aβ with or without PLGA as described earlier (67, 77, 78). The Native PAGE was performed using a 12% polyacrylamide gel at 4° C. using an Invitrogen novex Mini cell system. The gels were silver stained and visualized by a FluorChem E system (California, USA) and the images were processed by using Image J software.

Detection of Aβ₁₋₄₂ interaction with PLGA by epitope mapping: The site of Aβ₁₋₄₂ interacting with PLGA was detected by filter-trap assay using various site specific Aβ antibodies (see enclosed Table-1) (79). The Aβ samples after aggregation reaction in the presence or absence of PLGA were spotted on a nitrocellulose membrane on a nitrocellulose membrane (0.02 μm), subjected to vacuum filtration through a 96-well Bio-Dot Microfiltration system, washed with Tris-buffered saline and then incubated at 4° C. for 12 h with aggregate specific anti-Aβ OC antibody as well as different sequence-specific Aβ antibodies. The membranes were further washed with buffers, incubated with appropriate horseradish peroxidase-conjugated secondary antibodies (1:1000) and developed with an ECL kit. All blots were examined using a FluorChem E system (Santa Clara, Calif., USA) and the images were processed by using Image J software.

Mass spectroscopy (MS) analysis: To determine if 50 μM PLGA following 24-240 h interaction with 10 μM Aβ peptide is hydrolyzed into glycolic and lactic acids, MS analysis of lactic acid, glycolic acid and PLGA with or without Aβ₁₋₄₂ was carried out using an Agilent 6220 tandem mass spectrometer equipped with a FAB gun that produces a 6 keV xenon beam (Santa Clara, Calif., USA). Measurements were made in the negative-ion mode with glycerol as a matrix. The ion accelerating voltage was 10 kV and argon was used as a collision gas. MS/MS spectra were obtained by performing collision-induced dissociation (CID) in the third field-free region (3rd FFR) between MS-1 and MS-2. In the case of MS/MS/MS (MS3), 1st generation production generated from the precursor ion by CID in the first FFR was introduced to the 3rd FFR where CID was further performed. To the collision cell located in the 3rd FFR, a voltage corresponding to 30% of the kinetic energy of the selected ion was passed through MS-1.

Intracerebroventricular administration of native PLGA into 5×FAD and control mice: To determine the therapeutic potential of PLGA in attenuating AD pathology and behavior, we used 5×FAD mice which co-express three APP (Swedish mutation: K670N, M671L; Florida mutation: 1716V; London mutation: V7171) and two PS1 (M146L and L286V) FAD mutations and the age-matched non-transgenic (non-Tg), wild-type (WT) controls on C57BL/6J background. The phenotype and characteristic features of these 5×FAD mice were described previously (80, 81). These mice were purchased from The Jackson Laboratory (Bar Harbor, Me., USA) and housed under a standard a 12 h light/dark cycle with access to food and water ad libitum in accordance with Canadian Council on Animal Care guidelines. Three-month old 5×FAD along with age-matched non-transgenic wild-type mice (non-Tg/WT) were stereotaxically inserted with a microcannula into the right ventricle (−0.8 mm mid/lateral, −0.1 mm antero/posterior and −3.0 mm dorso/ventral from Bregma) under anesthesia and connected to an Alzet mini-osmotic pump (Model 2004) implanted subcutaneously on the back of the mouse. The pump infused either artificial cerebrospinal fluid (CSF) or unconjugated PLGA dissolved in CSF (at a constant flow rate of 0.25 μL/h) into the lateral ventricular pocket for 28 days. On the basis of volume and rate of synthesis/renewal of the mouse CSF, these conditions result in a concentration of 25 μM PLGA in the mouse CSF at equilibrium. Animals were monitored on a daily basis during 28 days of treatment for signs of weight loss and abnormal behavior. After treatment, animals were subjected to novel-object recognition test and then were either euthanized by decapitation or fixed by perfusion in 4% paraformaldehyde (PFA) for subsequent processing.

Western blotting: Western blotting was performed on CSF and PLGA treated 5×FAD and WT mice as well as control and Aβ-treated cultured neurons as described earlier (62, 63). In brief, brain tissues/cultured neurons from various experimental paradigms were homogenized with radioimmunoprecipitation lysis buffer and proteins were quantified using BCA kit. Denatured samples were resolved on 10% or 12% gradient sodium polyacrylamide gels or 4 to 12% NuPAGE Bis-Tris gels, transferred to PVDF membranes, blocked with 5% milk and incubated overnight at 4° C. with various primary antibodies at dilutions listed in Table-1. The membranes were then incubated with horseradish peroxidase-conjugated secondary antibodies (1:5000) and immunoreactive proteins were detected with ECL kit. All blots were re-probed with anti-β-actin antibody and quantified using ImageJ as described earlier (63).

Detection of oxidative stress: Control and Aβ-treated cortical cultured neurons with or without exposure to PLGA nanoparticles (100 or 200 μg/ml) were assayed for protein carbonyl content using the Oxyblot™ protein oxidation kit following the manufacturer's protocol. All blots were re-probed with anti-β-actin to monitor protein loading as described earlier (82).

Immunostaining: Cultured neurons grown on coverslips were first exposed to 100 μg/ml fluorescein-PLGA for 4 h, fixed in 4% PFA and processed for immunocytochemistry using anti-LAMP1 antibody to determine its localization on lysosomes. The immunostained sections were examined and photographed using a Zeiss confocal microscope (LSM 700, Carl Zeiss, Inc. Germany) equipped with a 63× Plan-apochromatic oil-immersion lens. In a parallel set of experiments, mouse cortical cultured neurons were treated with 10 μM Aβ₁₋₄₂ for 24 h in the presence or absence of 100 or 200 μg/ml PLGA and then exposed to the pH-sensitive LysoSensor Yellow/Blue DND-160, at 5 μM for 10 min (83). The fluorescent signal was measured with two emission images at 450 nm and 510 nm, both excited at 405/444 nm and then visualized under a Zeiss confocal microscope. With regard to 5×FAD mice, brain sections (20 μm) from the parietal cortex and cerebellum of CSF and PLGA-treated 5×FAD and WT mice were processed for immunostaining following the free-floating procedure for localization of antigens. The PFA fixed brain sections were incubated overnight at 4° C. with anti-Aβ OC antibody (dilutions in Table-1) and then processed as described earlier (82, 84). Immunostained brain sections were visualized and imaged using a Nikon Eclipse 90i fluorescence microscope equipped with a Retiga 2000R Q imaging system (Nikon Instruments Inc., NY, USA).

ELISA for Aβ₁₋₄₀ and Aβ₁₋₄₂: Aβ₁₋₄₀ and Aβ₁₋₄₂ levels in the parietal cortex and cerebellum of 5×FAD and WT control mice (4-6 mice/group) treated with CSF or PLGA were measured using commercially available ELISA kits as described earlier (82, 85). All samples were assayed in duplicate and each experiment was repeated 3 times.

Novel object recognition test: 5×FAD and WT control mice following 28 days administration of CSF or PLGA using mini-osmotic pumps were subjected to novel-object recognition memory test as described earlier (86). In brief, on day one, mice were habituated for five minutes in an open field—empty box followed by a day two familiarization phase, where the mice were exposed to two different objects (<10 min) placed in the box to get familiarize. On day three, among the two objects, one of the objects was replaced with a novel object and their exploratory behavior towards the familiar and novel object was quantified using a memory discrimination index (MI), wherein “tₒ” represents time exploring an object during the original exposure and “tₙ” represents time spent exploring an object that is novel on re-exposure: MI=(tₙ−tₒ)/(tₙ+tₒ). In parallel, the total number of visits to the novel object for all animals were evaluated.

Statistical analysis: All data collected from a minimum of 3-9 biological repeats with each experiment performed in at least three replicates were expressed as means±SEM. The cell viability data from cultured neurons were analyzed by one-way ANOVA followed by Bonferroni's post-hoc analysis for multiple comparisons with a significance threshold set at p<0.05. All statistical analysis was performed using GraphPad Prism (GraphPad Software, Inc., CA, USA).

### Results

A) PLGA Nanoparticles Protect Mouse Cortical Cultured Neurons Against an-Mediated Toxicity

A progressive increase in Aβ levels derived from increased production and/or decreased clearance is believed to underlie the cause of neurodegeneration and development of AD (2, 87-89). Consistent with this notion, a range of in vitro studies have shown that prolonged exposure to μM concentrations of Aβ peptide, depending on its β-sheet structure/fibrillar state, can cause neuronal toxicity (15, 88, 90). Apart from regulating intracellular signaling mechanisms underlying phosphorylation of tau protein, there is evidence that toxic concentrations of Aβ peptide can trigger lysosomal leakage, leading to release of the enzymes cathepsins B and D (CatB and CatD) into the cytosol. The enzymes can subsequently trigger cleavage of the Bcl2 family member Bid that promotes activation of Bax/Bak, which in turn initiates cytochrome c (Cyto c) release from mitochondria (91-97). Once in the cytosol, Cyto c associates with Apaf-1, forming an apoptosome complex that, in the presence of dATP/ATP, activates caspase-9 followed by caspase-3, leading to cell death (97-99). At present, however, the mechanisms by which Aβ peptide can trigger lysosomal leakage remains unclear as multiple agents/molecules can permeabilize the lysosomal membranes. Interestingly, generation of reactive oxygen species (ROS) has been shown to trigger lysosomal membrane permeability in many settings. Excess H₂O₂, generated from oxidative stress following Aβ treatment, diffuses into lysosomes to react with redox-active iron, resulting in the production of ROS (e.g., hydroxyl radicals) which subsequently trigger lysosomal leakage. In fact, this phenomenon has been described by us and others in a variety of cellular and animal models of neurodegeneration (61, 82, 88, 94, 95, 97, 100-103). Considering the evidence that PLGA nanoparticles can restore the function of compromised lysosomes (104), it would be of therapeutic relevance to determine if PLGA is able to protect neurons against Aβ-mediated toxicity. To address this issue, we used mouse primary cortical cultured neurons to evaluate the protective effect of PLGA against Aβ-induced toxicity.

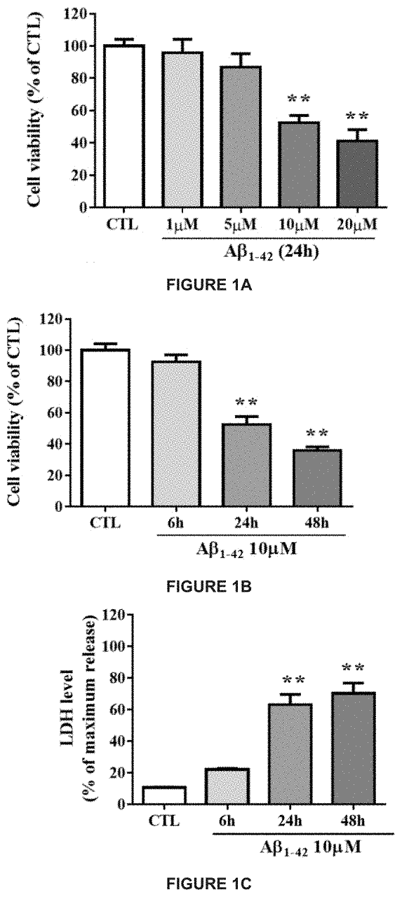

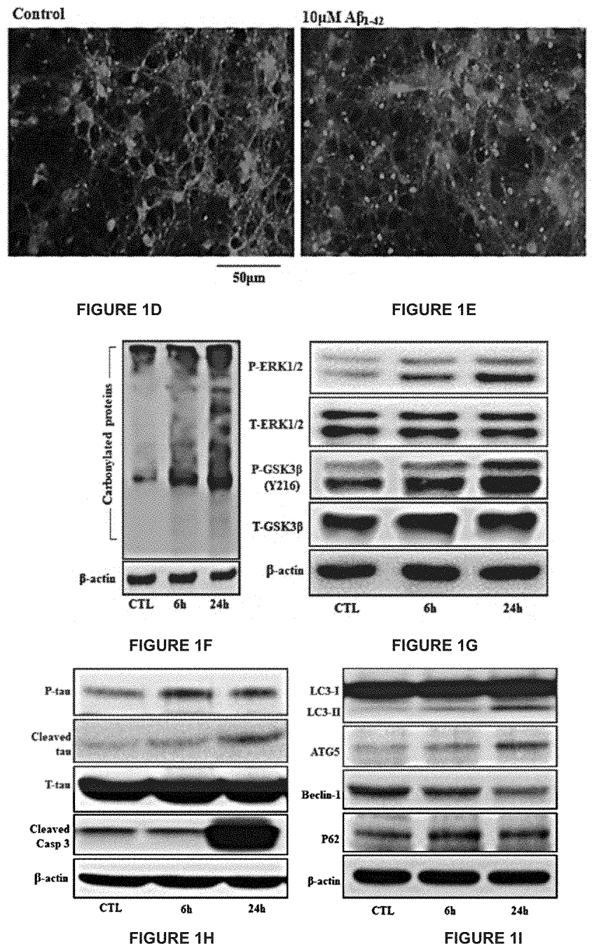

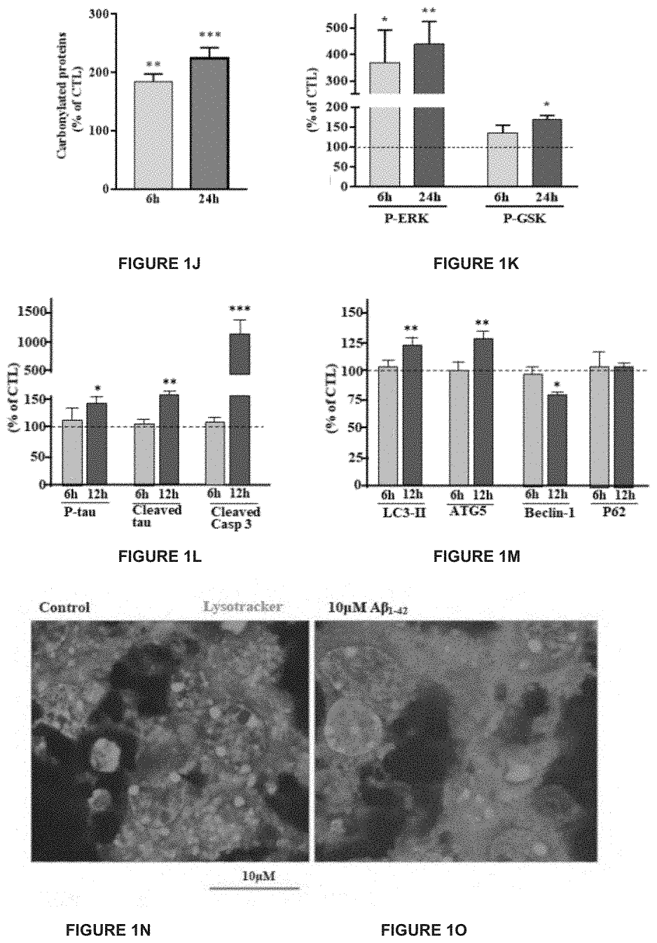

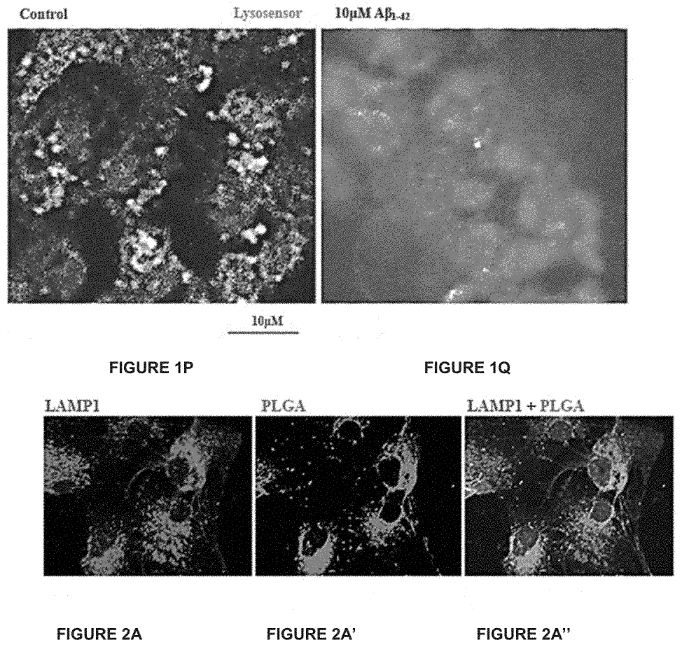

Characterization of Aβ-mediated toxicity: Mouse primary cortical cultured neurons are vulnerable to Aβ₁₋₄₂-mediated toxicity, as evident from a concentration- and time-dependent reduction in MTT values. The Aβ₄₂₋₁ sequence (i.e., control peptide), in contrast to the regular Aβ₁₋₄₂, did not alter MTT values, thus indicating the specificity of the effect (FIG. 1A, B). This was validated by a time-dependent increase in LDH levels in the conditioned media of the neurons treated with 10 μM Aβ₁₋₄₂ (FIG. 1C). The live/dead cell assay also revealed that exposure to 10 μM Aβ₁₋₄₂ for 24 h can induce a marked increase in the number of dead neurons (FIG. 1D, E). In keeping with earlier studies (87, 103, 105, 106), we observed that Aβ₁₋₄₂-induced toxicity was associated with increased levels of oxidative stress marker carbonylated proteins (FIG. 1F, J), enhanced levels of phosho-Thr²⁰²/Tyr²⁰⁴ extracellular-signal related kinase 1/2 (ERK1/2), phospho-Tyr²¹⁶ glycogen synthase kinase (GSK-3β) (FIG. 1G, K), phospho-tau, cleaved-tau and cleaved caspase-3 (FIG. 1H, L). Also, Aβ₁₋₄₂ toxicity is accompanied by marked increases in the levels of autophagic markers LC3-II and autophagy protein 5 (ATG5), unaltered levels of P62 and decreased levels of beclin-1 (FIG. 1I, M). LysoTracker red DND-99 is a weak basic amine that exhibits punctate labelling following its selective accumulation in cellular compartments with low internal pH such as lysosomes (107). Our results revealed a punctate labelling of LysoTracker red DND-99 in control but not in Aβ-treated neurons (FIG. 1N, O) suggesting a compromise in lysosomal integrity following Aβ treatment. This is partly supported by our staining with LysoSensor yellow/blue DND-160. This reagent, engineered to detect pH changes in a broad range (pKa ˜4.2, <4.2 yellow, >4.2 blue) including lysosomal pH (107) displayed an acidic environment by yellow fluorescence in the control neurons, whereas Aβ-treated neurons exhibited a somewhat basic environment by blue fluorescence reflecting leakage/breakdown of lysosomes (FIG. 1P, Q).

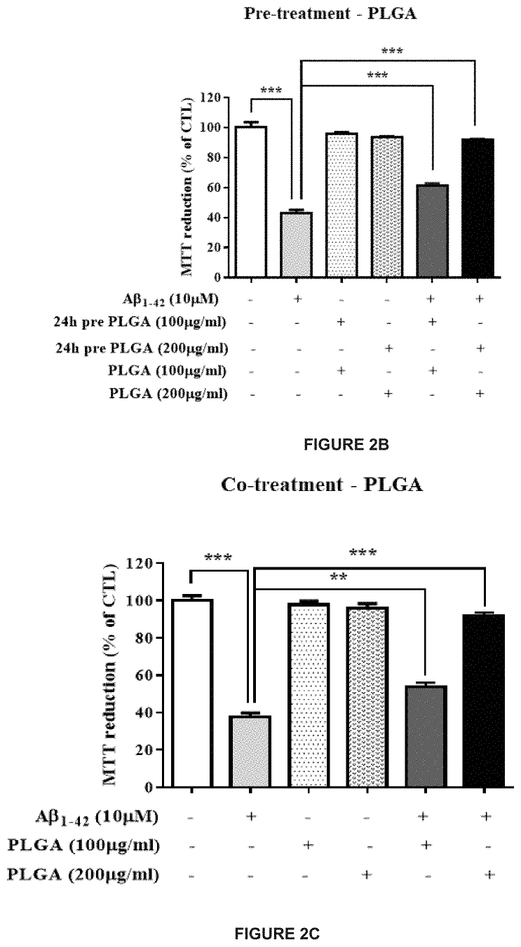

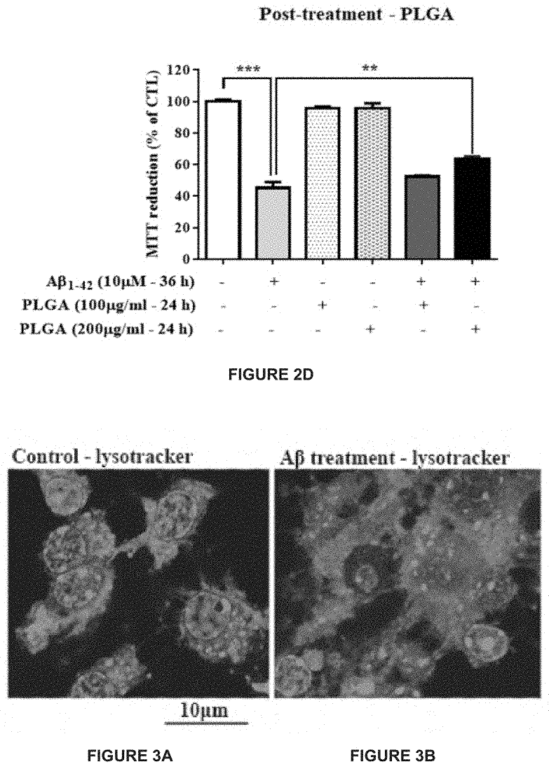

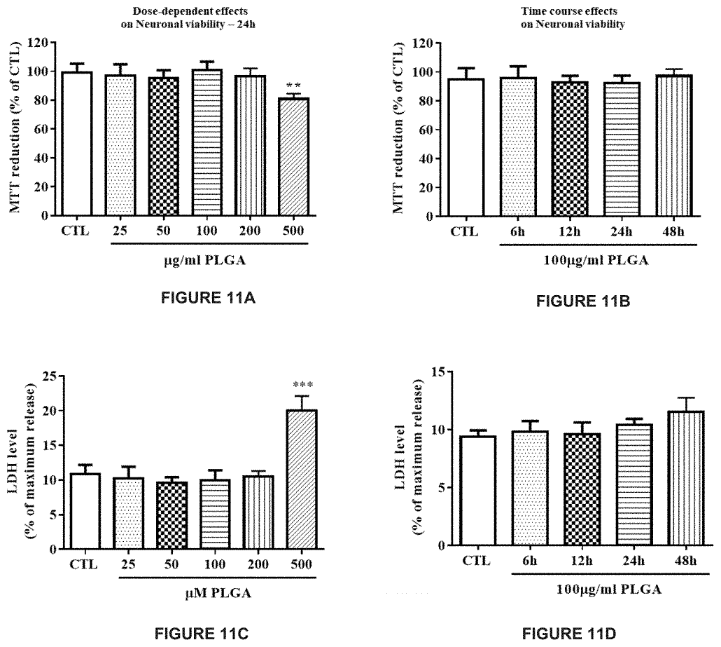

Protective effects of PLGA against Aβ-mediated toxicity: Some recent studies have shown that PLGA encapsulated drugs/agents such as donepezil, memantine and curcumin can have beneficial effects on cellular or animal models of AD (47-50, 53-55). However, it remains unclear if native unconjugated PLGA can protect neurons against Aβ-mediated toxicity by restoring lysosomal leakage. To address this issue, we first demonstrated that fluorescent PLGA following internalization into cultured neurons is targeted primarily to lysosomes as evident from its co-localization with lysosomal marker LAMP1 (FIG. 2A, A′, A″). Additionally, we observed that 100 or 200 μg/ml native PLGA did not exhibit any toxic effect following 24 h exposure to cultured neurons, whereas 500 μg/ml PLGA reduced neuronal viability after 24 h exposure (FIG. 11). Thus, cortical neurons were treated with 100 or 200 μg/ml native PLGA for 24 h prior to exposure with 10 μM Aβ₁₋₄₂ and then neuronal viability was assessed following 24 h using the MTT assay. Interestingly, both 100 and 200 μg/ml PLGA were found to protect neurons against Aβ-induced toxicity (FIG. 2B). Concurrent treatment of neurons with 100 or 200 μg/ml PLGA along with 10 μM Aβ₁₋₄₂ for 24 h was able to significantly protect neurons against toxicity (FIG. 2C). To determine the rescuing property of PLGA, neurons were first exposed to 10 μM Aβ₁₋₄₂ for 12 h then treated with 100 or 200 μg/ml PLGA for an additional 24 h. Interestingly, 200 μg/ml, but not 100 μg/ml PLGA, was able to rescue neurons significantly following 12 h exposure to Aβ₁₋₄₂ (FIG. 2D). Our data also reveal that 100 and 200 μg/ml PLGA can protect cultured neurons derived from induced pluripotent stem cells (iPSC) of control and AD patients against Aβ₁₋₄₂-induced toxicity (data not shown).

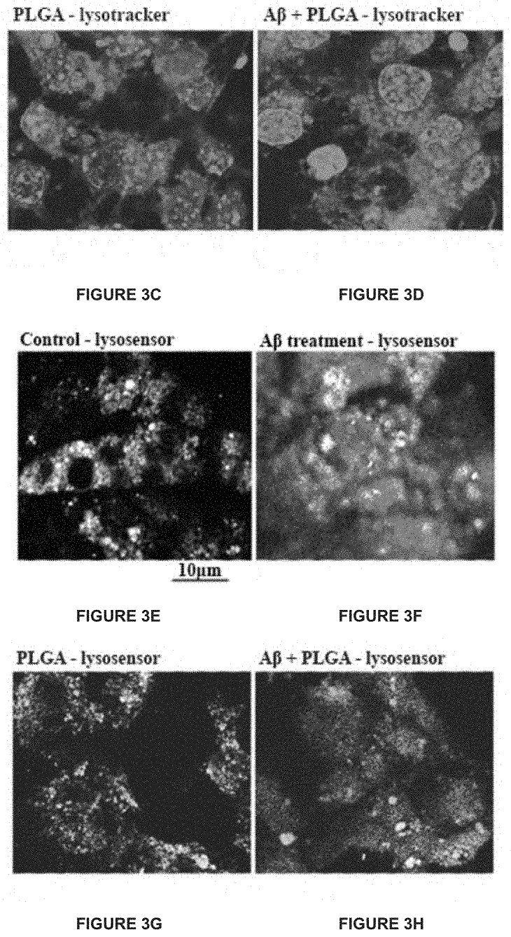

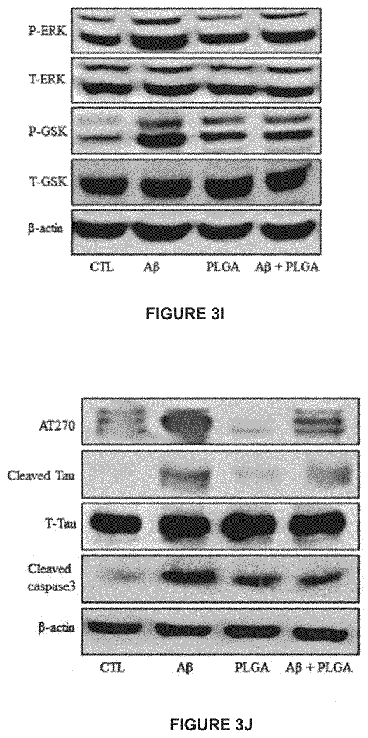

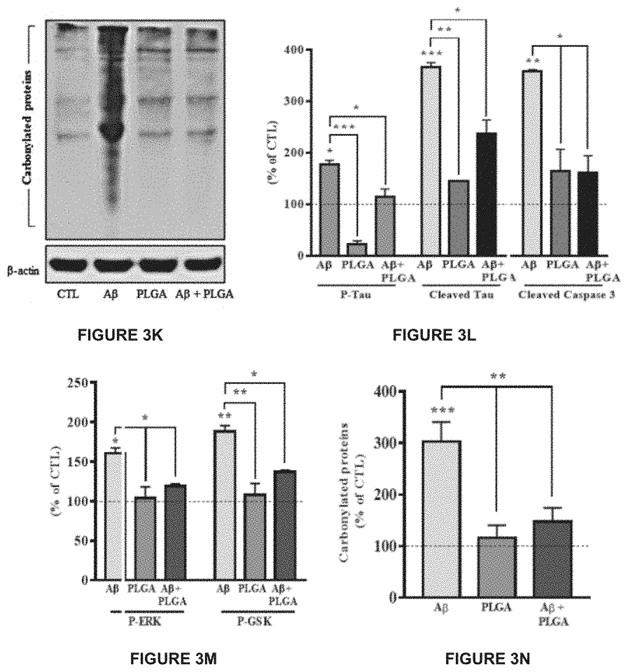

Our LysoTracker labelling revealed that PLGA treatment decreased diffuse labelling of cultured neurons observed following Aβ treatment (FIG. 3A-D). This is supported by our LysoSensor yellow/blue DND-160 labelling which displayed a more acidic environment in Aβ+PLGA-treated neurons, as observed in control and PLGA alone treated conditions, than the basic environment detected in Aβ-treated cultured neurons (FIG. 3E-H). In parallel, we noted that the protective effects of PLGA against Aβ toxicity are associated with an attenuation of ERK1/2 and GSK-3β activation (FIG. 3I, L), decreased levels of phosphorylated and cleaved tau protein as well as cleaved caspase-3 (FIG. 3J, M). Additionally, PLGA significantly reduced the levels of protein carbonyl groups in Aβ-treated neurons, suggesting that its protective effect may partly be mediated by attenuating ROS production (FIG. 3K, N) which plays an important role in triggering lysosomal membrane permeability.

B) Native PLGA Nanoparticles Attenuate In Vitro Aggregation and Toxicity of Aβ Peptide

Formation of Aβ fibrils is a key event in AD pathogenesis. Monomers of Aβ₁₋₄₂ can form soluble aggregates ranging from dimers to dodecamers that can further oligomerize to form aggregates of higher-order including fibrils (108-111). Structurally, amyloid fibrils are aggregates with repetitive cross-beta sheets stabilized by different molecular interactions. Interestingly, the ongoing aggregation process per se is associated with cellular toxicity as soluble oligomers of different sizes, starting with dimers, are considered more toxic as well as more pathogenic than either monomers or mature fibrils. Thus, many studies have pursued small organic molecules, peptides and nanoparticles that can prevent Aβ aggregation and/or toxicity as a treatment strategy for AD (104, 109-115). However, most nanoparticles employed so far in AD pathology have been functionalized with inhibitors such as curcumin, resveratrol, deferoxamine, D-penicillamine or clioquinol to prevent Aβ aggregation by improving their permeability across the BBB, binding affinity and/or specificity with Aβ peptide (29-44, 94, 102, 108). To the best of our knowledge native polymeric nanoparticles have not yet been shown to directly inhibit Aβ aggregation and/or cell toxicity.

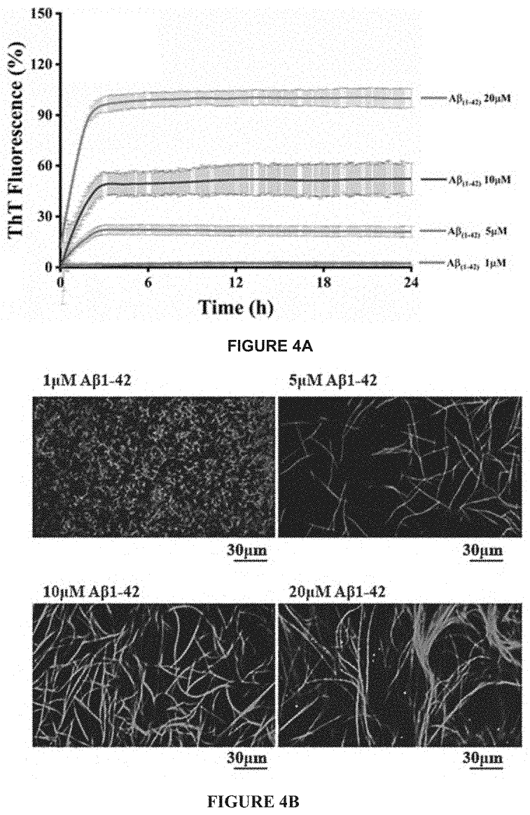

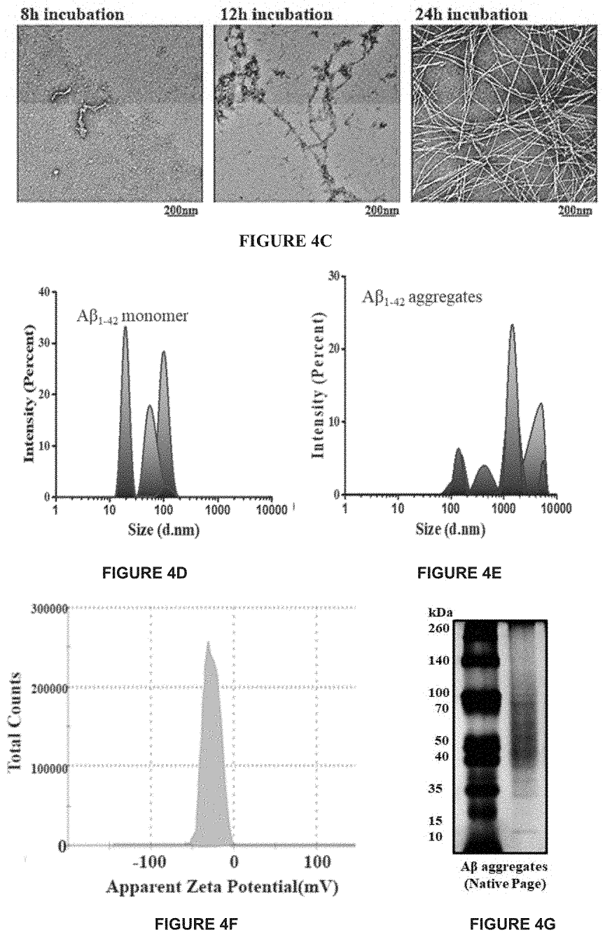

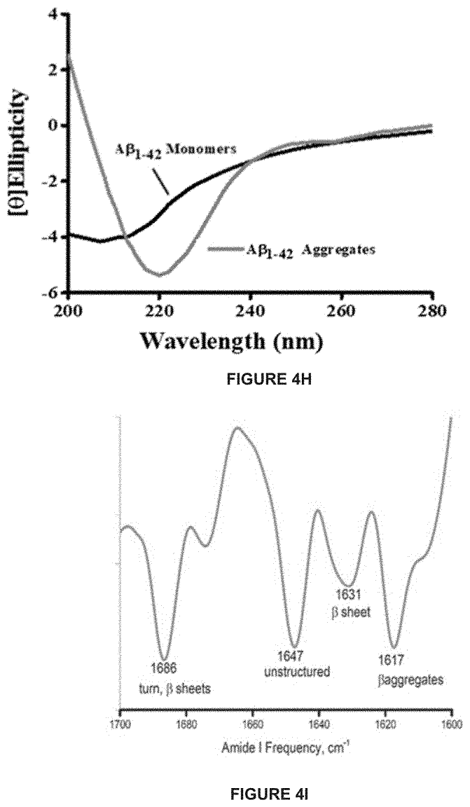

Characterization of spontaneous Aβ₁₋₄₂ aggregation: Our aggregation kinetic studies revealed that 1-20 μM Aβ₁₋₄₂, as expected, dose-dependently increased peptide aggregation over 6 h and then reached a plateau as indicated by enhanced ThT levels (FIG. 4A). The propensity of Aβ aggregation was validated by fluorescence imaging which showed a dose-dependent increased presence of Aβ fibrillar entities following ThT labelling (FIG. 4B). Our TEM studies also displayed the large twisted fibrillar entities following aggregation of Aβ₁₋₄₂ (FIG. 4C). The conversion of Aβ₁₋₄₂ from its monomeric to fibrillar state was substantiated by DLS which showed the presence of Aβ peptides with increased hydrodynamic radii ˜10-100 nm to ˜100-10,000 nm over 24 h incubation with the surface charge of −32 mV (FIG. 4D-F). Our native PAGE also revealed the presence of higher order entities of the aggregated Aβ following 24 h incubation by displaying a smeared band (FIG. 4G). To confirm the structural modifications before and after aggregation, we performed CD and FTIR spectrometry analysis using 10 μM Aβ₁₋₄₂. The obtained CD spectra clearly revealed the presence of coiled structures prior to aggregation (0 h), whereas following 24 h aggregation a structural shift towards beta structure was clear (FIG. 4H). Our FTIR spectrometry analysis revealed that secondary derivate spectra generated through the ATR mode depicted the characteristic vibrations at 1617, 1631, 1647, 1667, and 1684 cm⁻¹ indicating the presence of β-sheet enriched structures in Aβ₁₋₄₂ aggregates (FIG. 4I) (66, 76, 116).

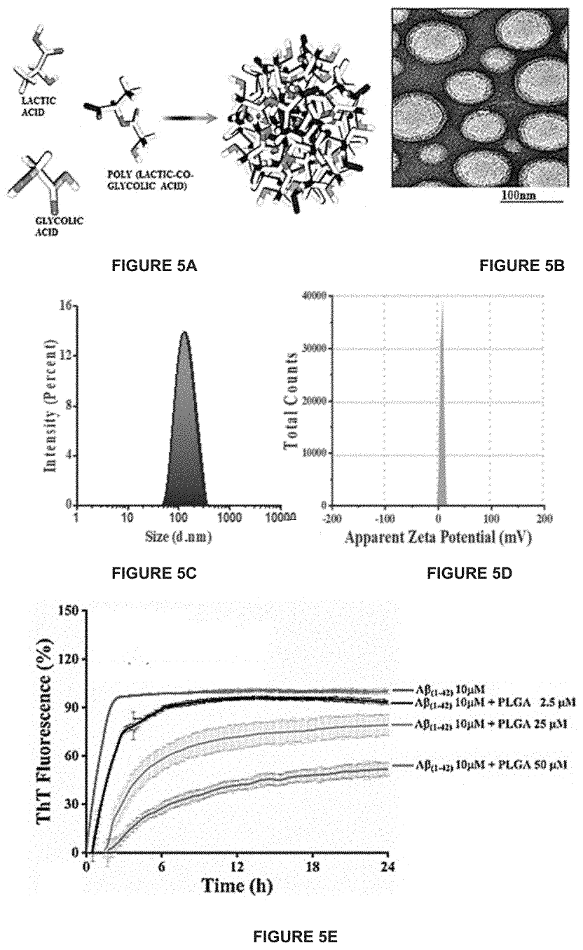

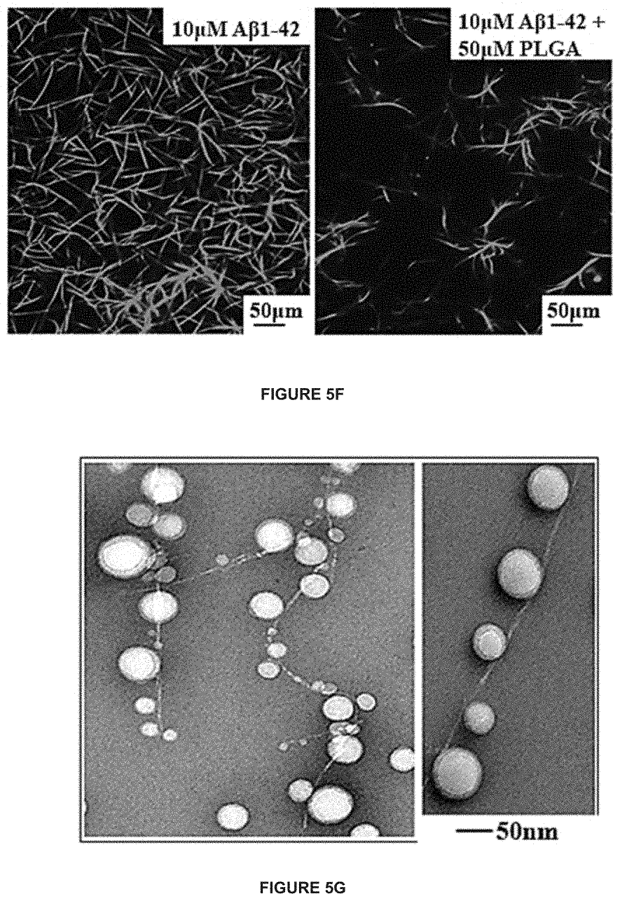

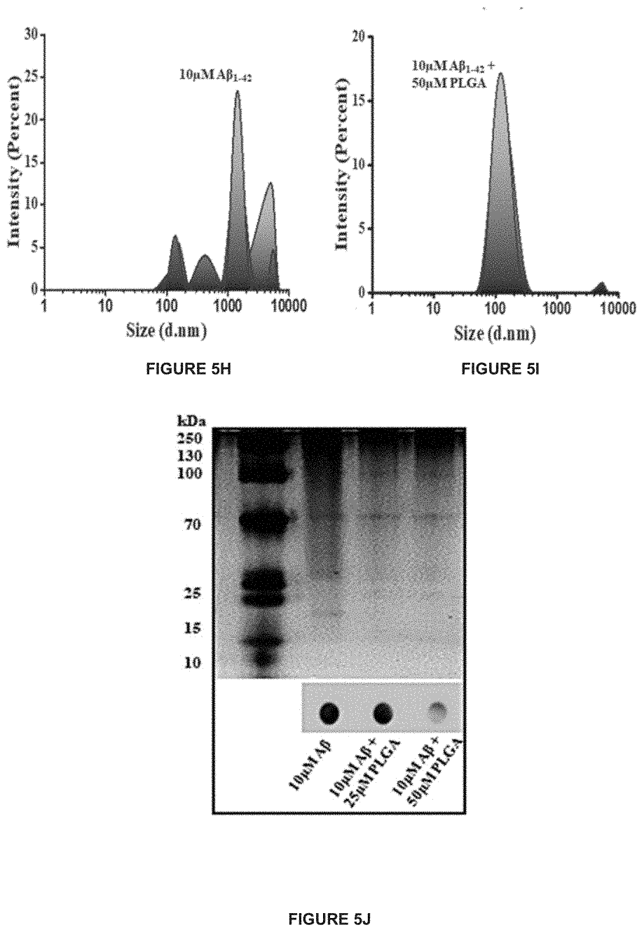

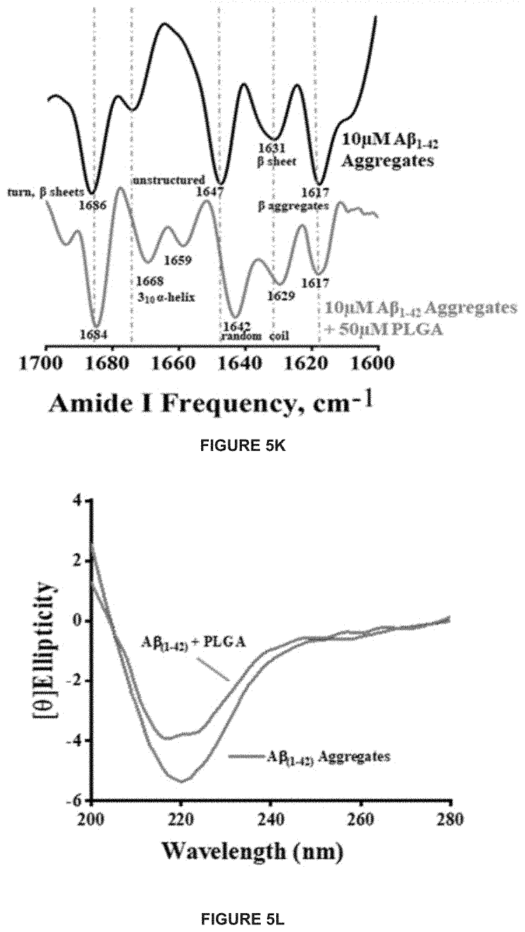

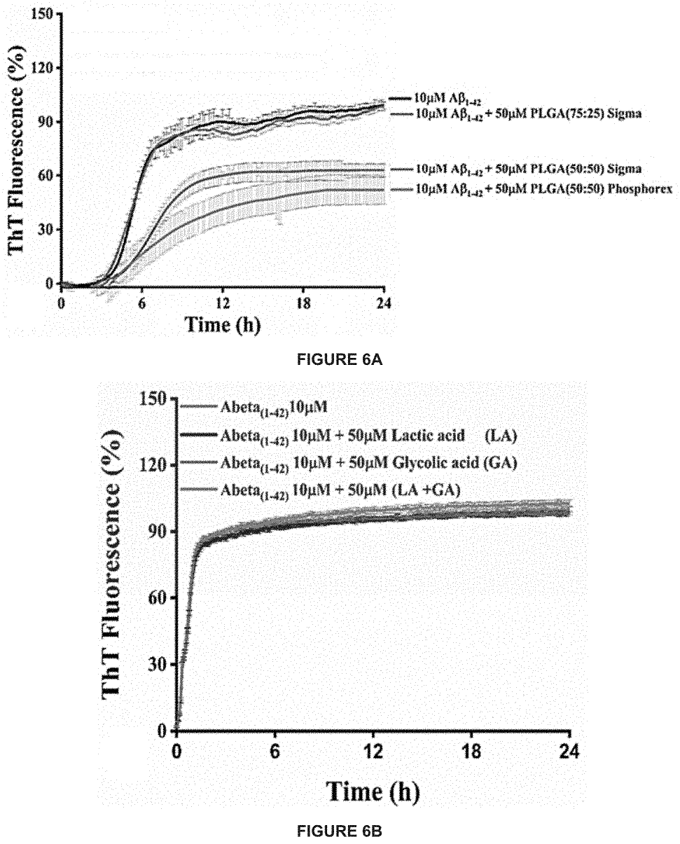

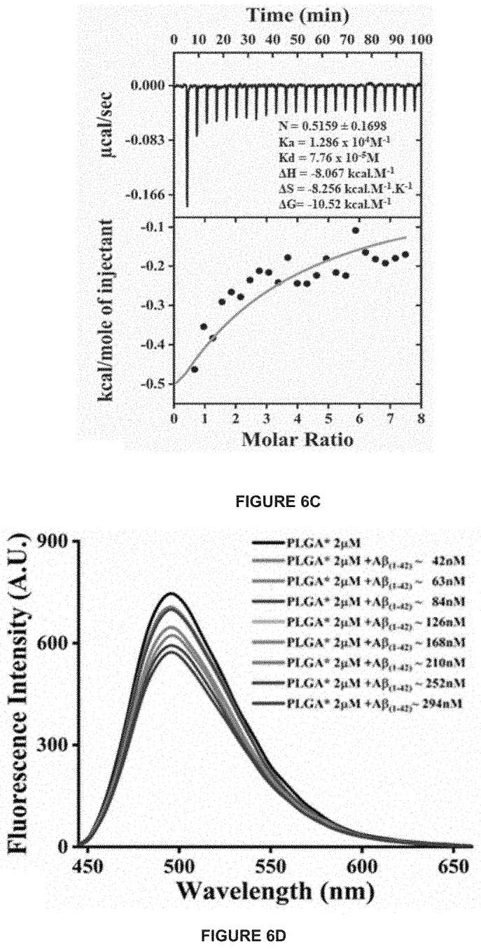

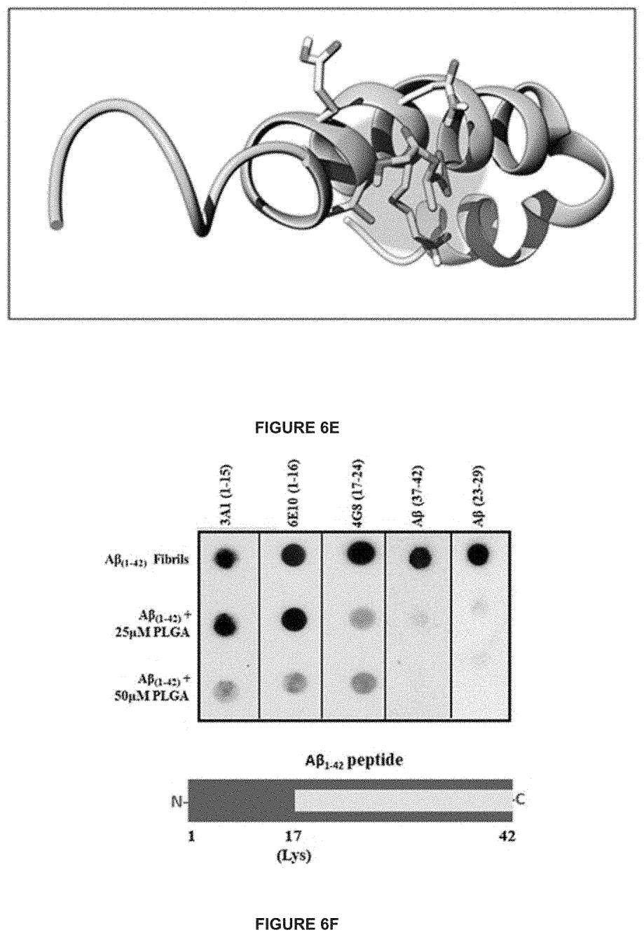

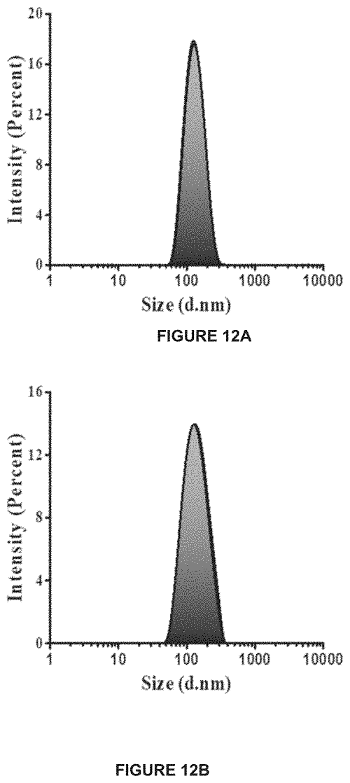

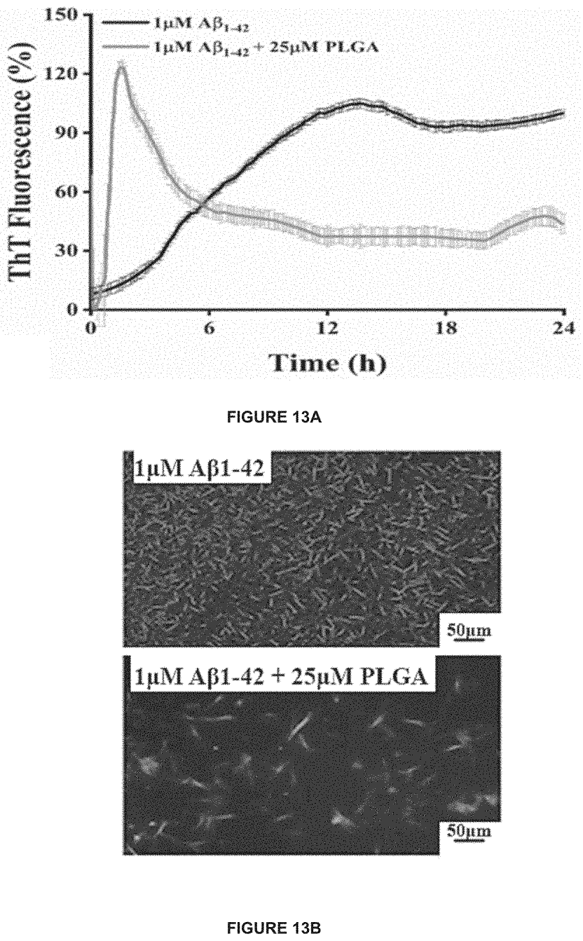

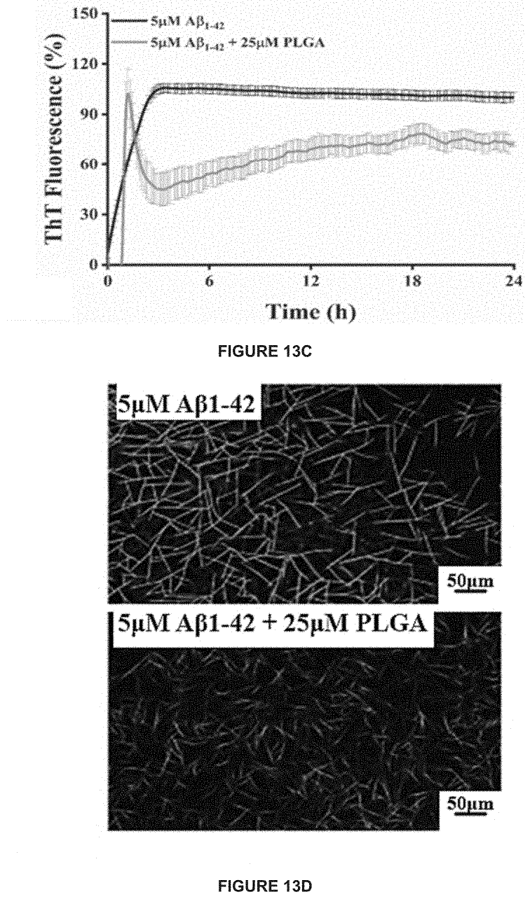

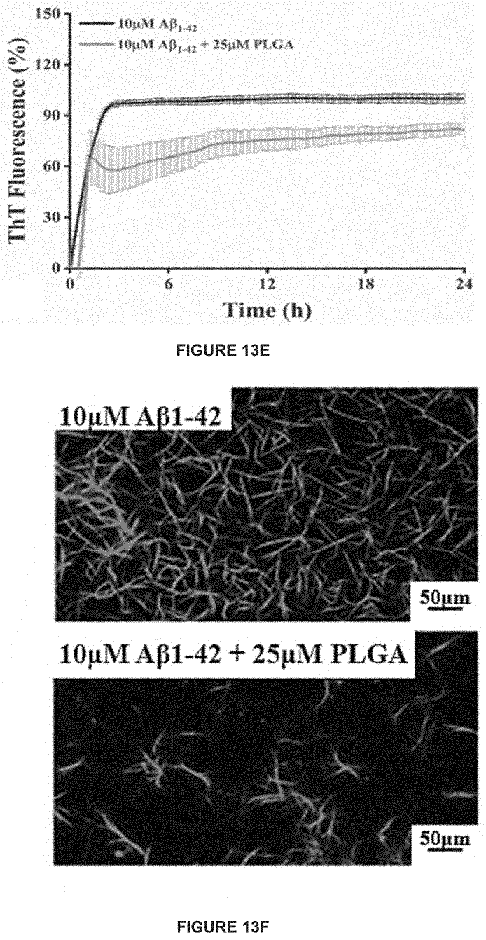

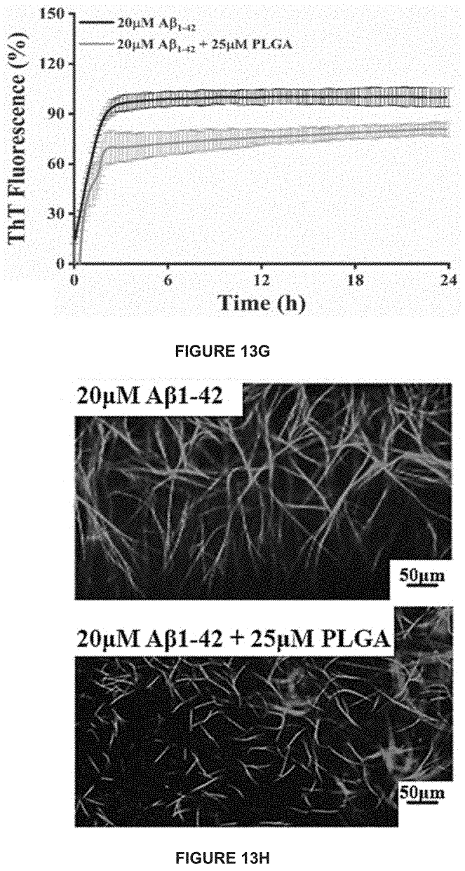

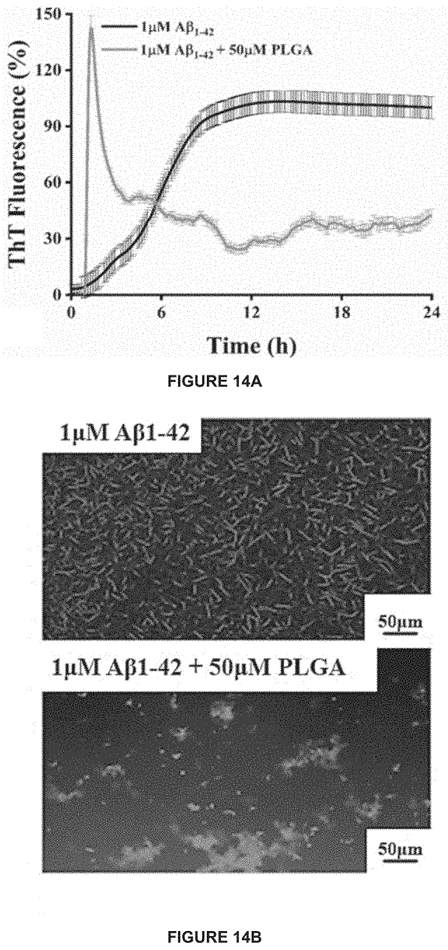

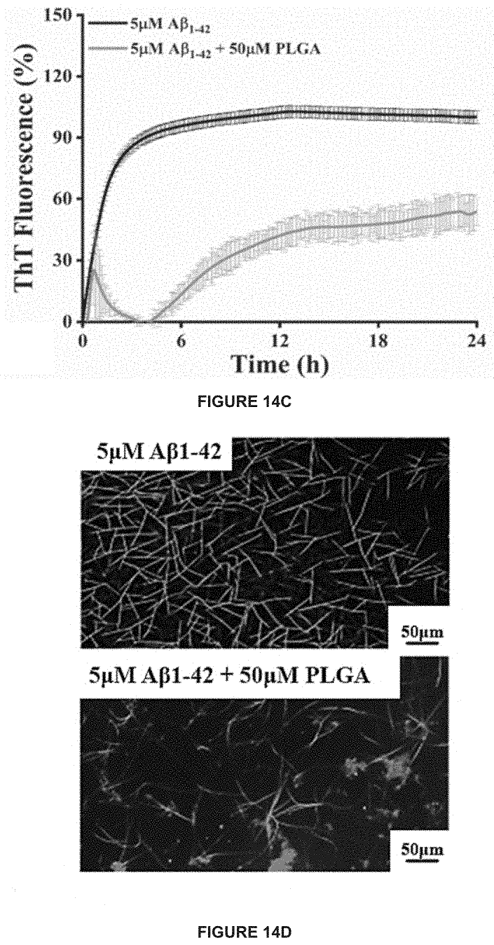

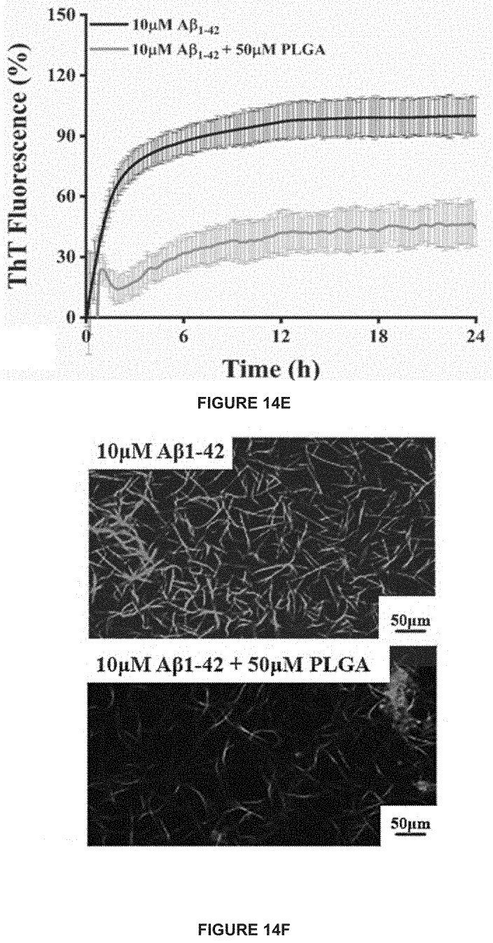

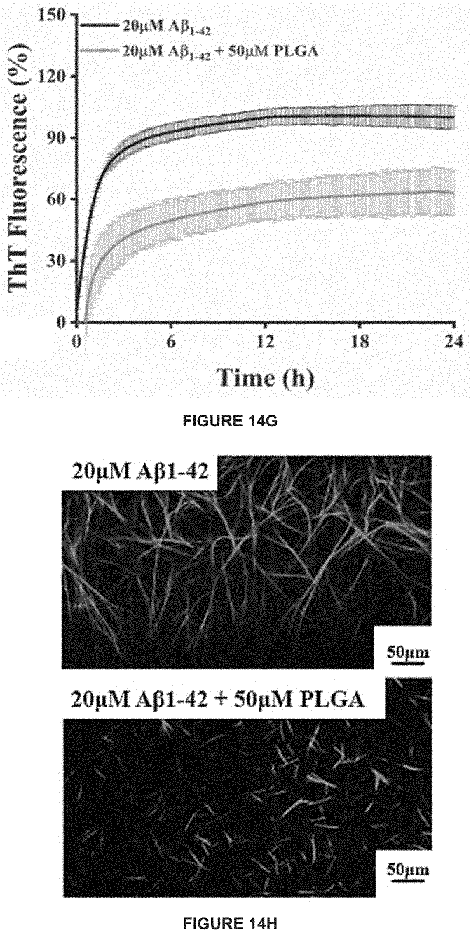

Attenuation of spontaneous Aβ₁₋₄₂ aggregation by PLGA: To determine if native PLGA can attenuate spontaneous Aβ aggregation, we first established the characteristic features associated with native PLGA (FIG. 5A). Our TEM data clearly show that PLGA nanoparticles are mostly homogeneous with spheroidal morphology with an average diameter of ˜100 nm, while a few small particles could be observed (FIG. 5B). DLS analysis also displays that PLGA is quite stable over a 240 h period with hydrodynamic radii ˜100 nm and the Zeta potential measurement depicting the surface charge of ˜8 mV (FIG. 5C, D; FIG. 12). Subsequently, we evaluated the aggregation kinetics of 10 μM Aβ₁₋₄₂ in the presence or absence of 2.5-50 μM unconjugated PLGA in phosphate buffer at 37° C. over a 24 h period using the ThT fluorescence assay. Our data clearly indicate that PLGA can dose-dependently attenuate Aβ₁₋₄₂ aggregation, possibly due to alteration in secondary nucleation leading to stabilizing the monomeric state of the Aβ₁₋₄₂ peptide (FIG. 5E). The attenuation of Aβ₁₋₄₂ aggregation is validated by fluorescence imaging which showed fewer Aβ fibrillar entities in presence of PLGA (FIG. 5F). Our TEM data also reveal that PLGA nanoparticles are directly associated with Aβ₁₋₄₂ possibly attenuating peptide aggregation leading to the formation of a heterogenous mixture of smaller Aβ aggregates (FIG. 5G). The transformation of Aβ₁₋₄₂ fibers to lower ordered entities by PLGA was evident in DLS as well as native PAGE analysis (FIG. 5H-J). While DLS data revealed that the hydrodynamic radii of Aβ₁₋₄₂ aggregates reduced markedly in the presence of PLGA (FIG. 5H, I), native-PAGE revealed the lack of higher-ordered aggregates in Aβ samples treated with PLGA (FIG. 5J). Furthermore, a filter-trap assay using a fibril-specific anti-Aβ OC antibody showed decreased formation of Aβ₁₋₄₂ fibers in presence of PLGA (FIG. 5J inset). CD spectroscopy revealed an increase in the helical content from 2.3 to 6.6% and a decrease in the β-sheet content from 38.6 to 28.8% in Aβ₁₋₄₂ samples treated with PLGA, suggesting an attenuation of the conformational transition of Aβ₁₋₄₂ from random coils to the β-sheets. Likewise, FTIR data showed the vibration of a 3₁₀ α-helix in the presence of PLGA rather than the β-sheet-enriched structures observed in Aβ₁₋₄₂ aggregates—implying retention of the monomeric state (FIG. 5K, L). To further substantiate the inhibitory effects of PLGA, we also measured the aggregation kinetics of 1-20 μM Aβ₁₋₄₂ in the presence or absence of 25 or 50 μM PLGA over a 24 h period. Our ThT kinetic data as well as fluorescence imaging clearly show that aggregation of various concentrations of Aβ₁₋₄₂ was markedly attenuated in presence of 25 and 50 μM PLGA as a function of time (FIGS. 13, 14). Our preliminary data also reveal that 2.5-50 μM PLGA over 24 h period can suppress spontaneous aggregation of 4-repeat tau protein (10 μM)—which plays an important role in the formation of neurofibrillary tangles in AD brains (data not shown). Finally, to establish the specificity of the PLGAs' effect, we first showed that PLGA with 50:50 resomer (red color in FIG. 6A) from a different source (i.e., Sigma-Aldrich) was able to suppress spontaneous Aβ₁₋₄₂ aggregation, whereas equimolar PLGA with 75:25 resomer (purple color in FIG. 6A) compositions did not attenuate Aβ aggregation. In parallel, we showed that aggregation of Aβ₁₋₄₂, as revealed by our ThT kinetic assay, was not inhibited either by 50 μM lactic acid, 50 μM glycolic acid or a mixture of 50 μM lactic acid and glycolic acid, but only after polymerization of lactide and glycolide to PLGA (FIG. 6B).

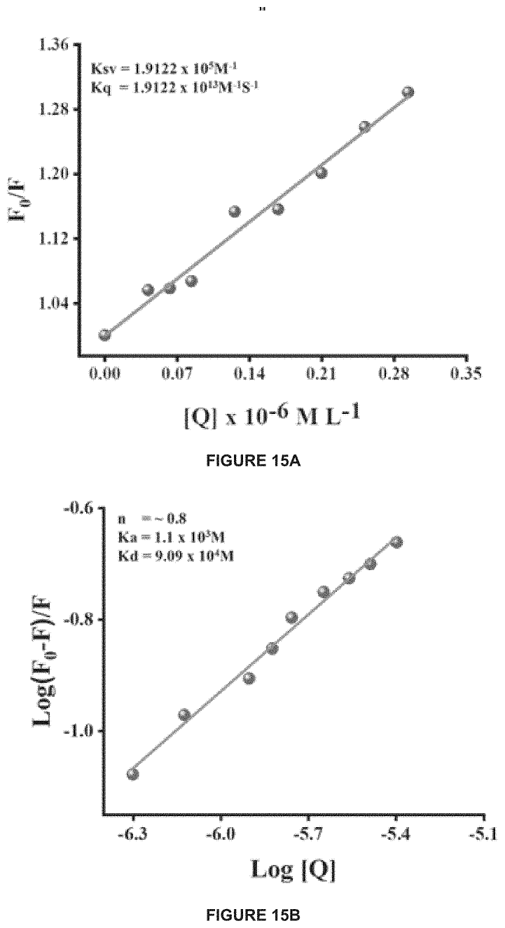

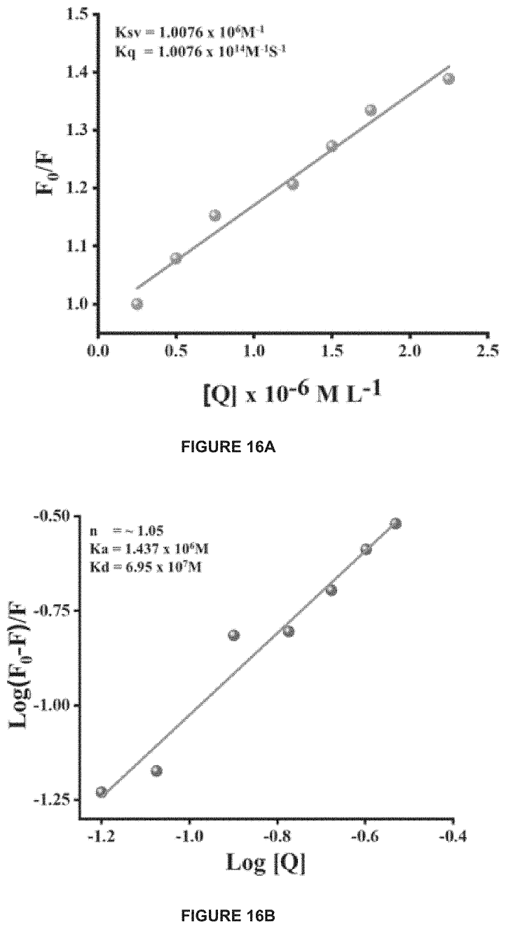

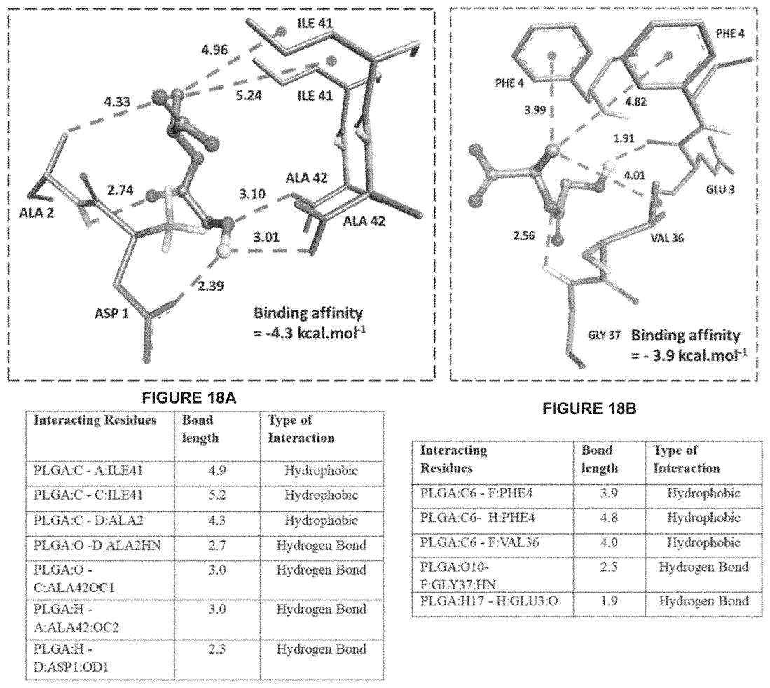

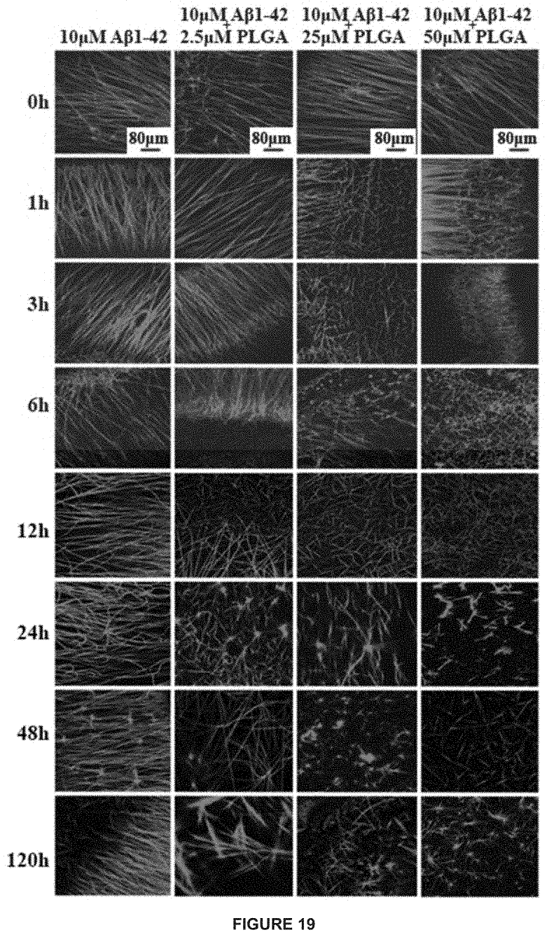

Molecular interaction of PLGA with Aβ₁₋₄₂ interaction: Our ITC experiments involving titration of PLGA to Aβ₁₋₄₂ showed an exothermic interaction with a Kd=7.76×10⁻⁵M, Ka=1.286 10⁴M⁻¹ and a stoichiometry of ˜1 (n=0.519). The Gibbs free energy ΔG was found to be −10.52 kcal·M⁻¹ with an Enthalpy (ΔH)=−8.067 kcal·M⁻¹ and an Entropy (ΔS)=−8.256 kcal·M⁻¹K⁻¹. In parallel, the fluorescence quenching revealed that the intrinsic fluorescence emission from PLGA was effectively quenched in a dose-dependent manner in the presence of monomeric Aβ₁₋₄₂ predicting a single binding site (n=0.8) with a Kd of 9.09×10⁴M for interactions between PLGA and Aβ(FIG. 6C, D; FIGS. 15-17). To understand better the molecular interaction, we subsequently performed molecular docking analysis which indicated that PLGA was able to interact with the region spanning from Lysine 17 to Alanine 42—known as the key aggregation prone domain in Aβ₁₋₄₂ (PDB ID:1IYT) with an interaction energy of 4.2 Cal/mol (FIG. 6E; FIGS. 18, 19). This is reinforced by our epitope mapping analysis of Aβ₁₋₄₂ samples in the presence or absence of PLGA using various isoform specific Aβ antibodies (FIG. 6F).

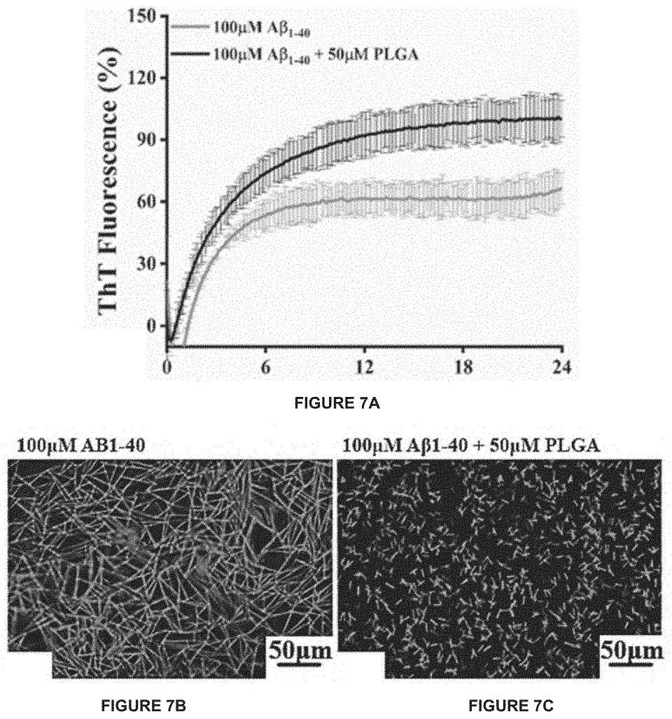

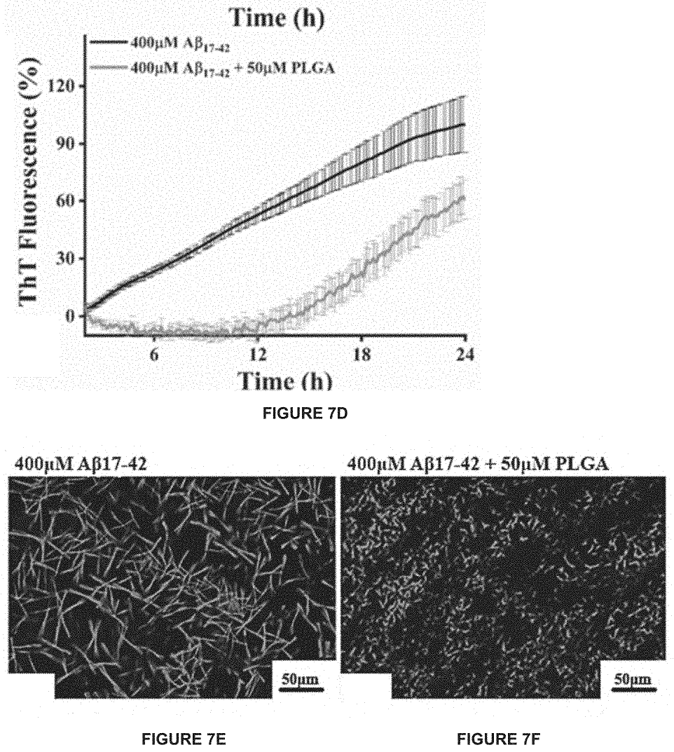

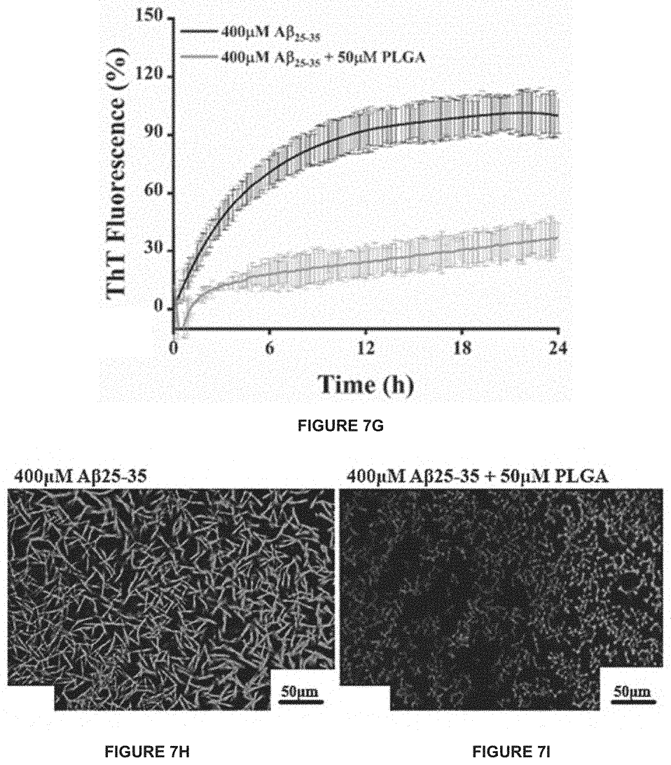

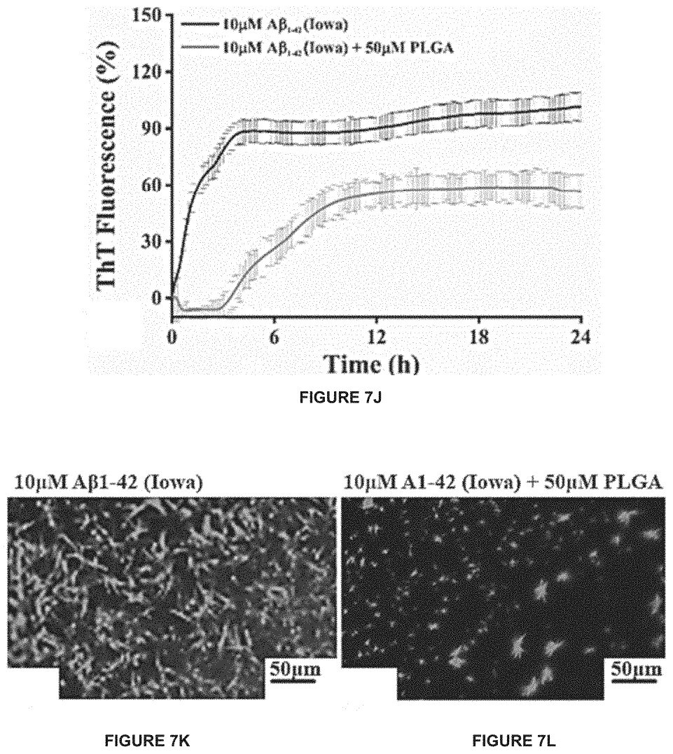

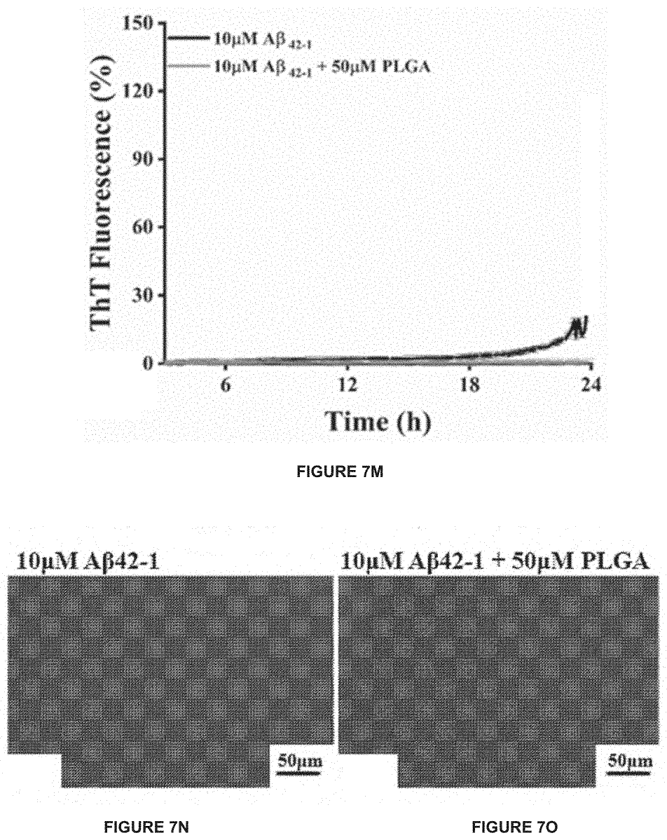

Specificity of PLGA and Aβ interactions: To determine if PLGA, apart from Aβ₁₋₄₂, can attenuate aggregation of other isoforms of Aβ peptide, we performed ThT aggregation assays using 10 μM Aβ₁₋₄₀, Aβ₁₇₋₄₂ and Aβ₂₅₋₃₅ in the presence or absence of 50 μM PLGA at 37° C. over a 24 h period (FIG. 7A-I). Our kinetic data as well as fluorescence imaging clearly revealed that PLGA, as observed with Aβ₁₋₄₂, can time-dependently suppress aggregation of all isoform/fragments of Aβ peptides. In parallel, we also measured the effects of PLGA on familial D23N Iowa mutant Aβ₁₋₄₂ which is known to aggregate faster and more toxic to neurons than normal Aβ₁₋₄₂ (117). Interestingly, 50 μM PLGA is able to decrease spontaneous aggregation of 10 μM D23N mutant Aβ₁₋₄₂—thus indicating that PLGA may be beneficial not only in sporadic but also in familial AD cases (FIG. 7J-L). It is of interest to note that 10 μM Aβ₄₂₋₁ (i.e., a negative control) which did not follow the aggregation kinetics of normal Aβ₁₋₄₂ was not affected by PLGA (FIG. 7M-O).

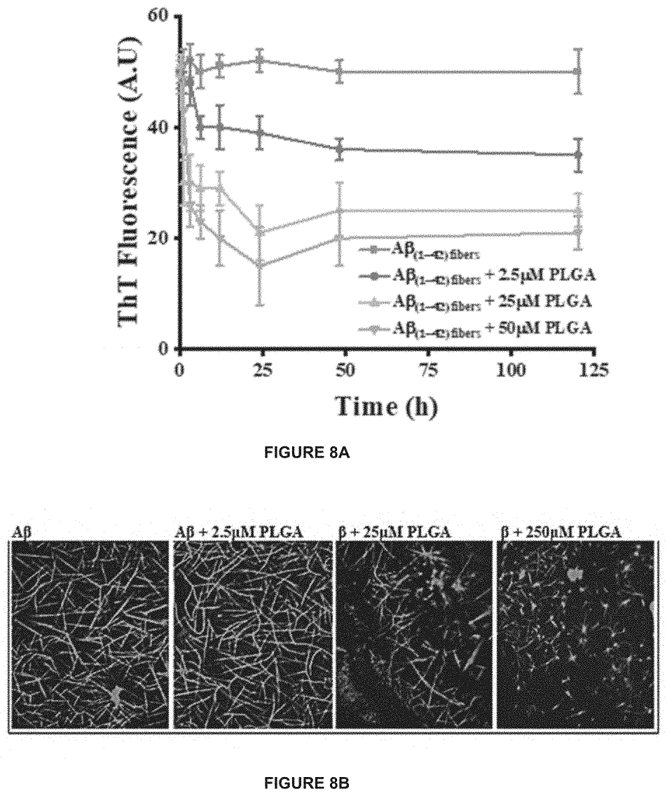

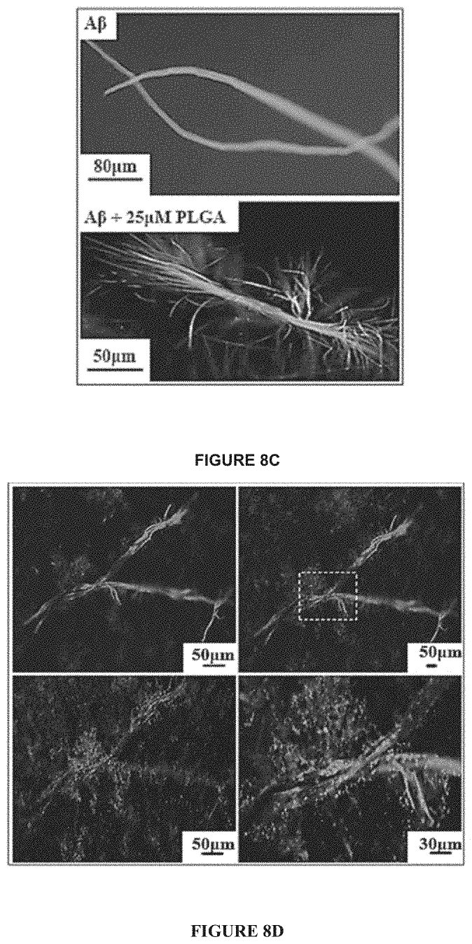

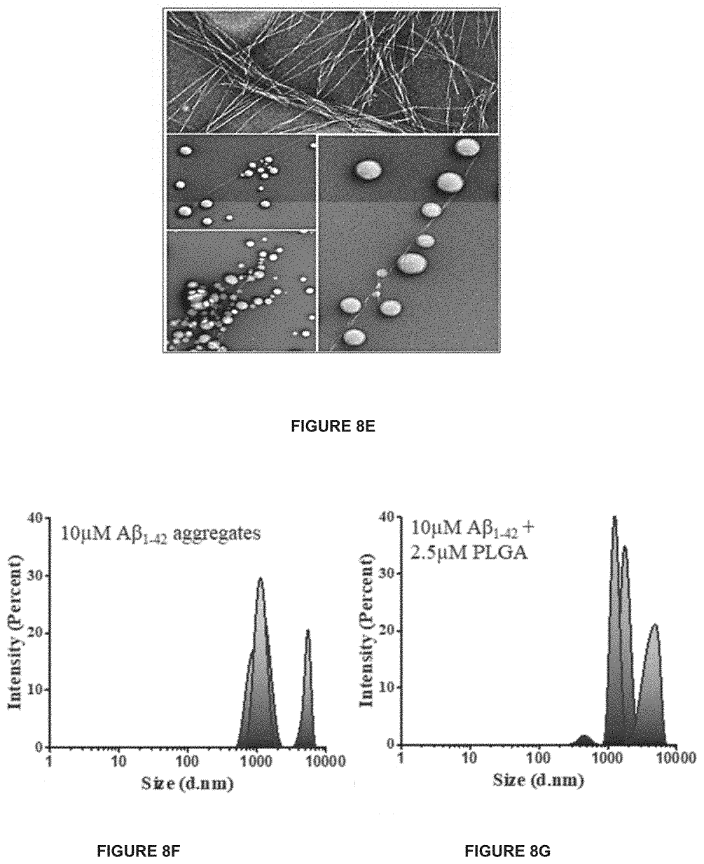

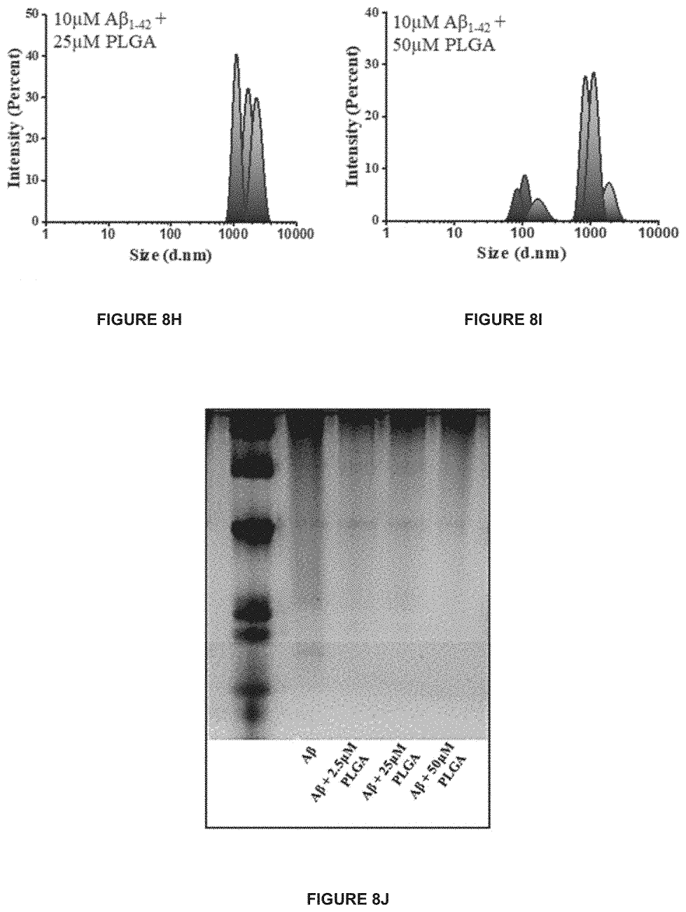

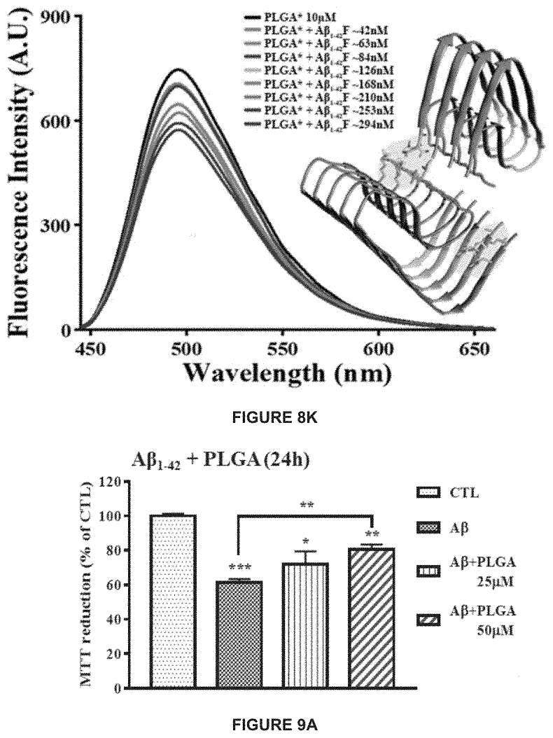

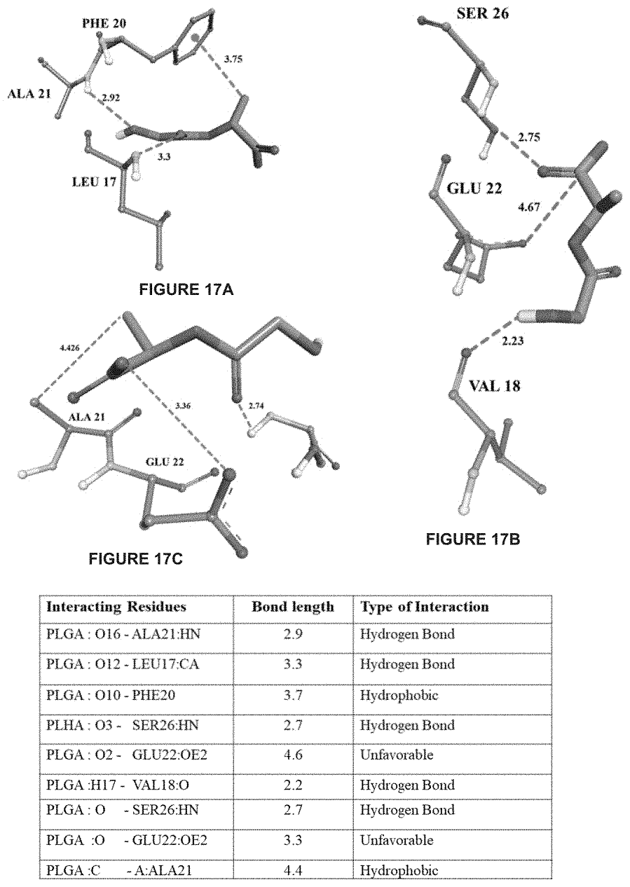

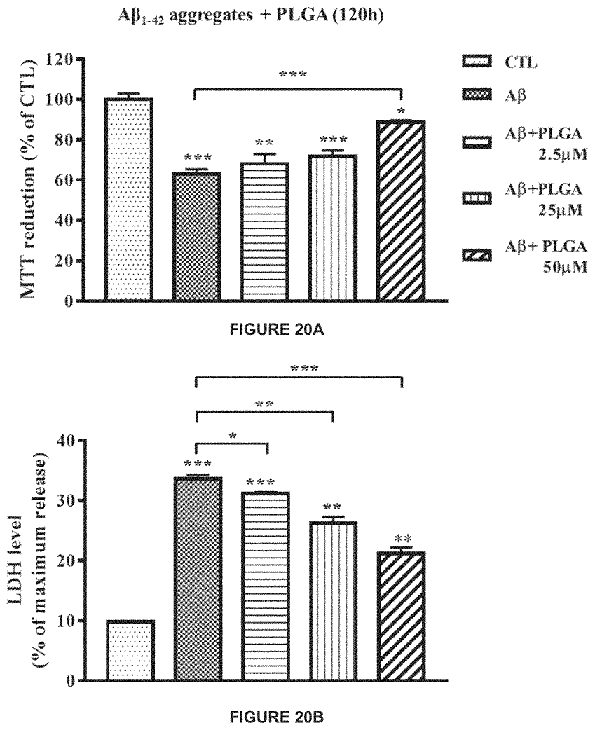

Disassembly of aggregated Aβ₁₋₄₂ fibers by PLGA: To highlight the therapeutic potential of PLGA in AD pathology, we incubated preformed Aβ₁₋₄₂ fibers with 2.5-50 μM native PLGA at 37° C. over 120 h. PLGA nanoparticles, as revealed using the ThT fluorescence assay, can time- and dose-dependently trigger disassembly of matured Aβ₁₋₄₂ fibers into small fibrillary entities possibly due to disruption of β-sheet structure or interaction with the steric zippers present in Aβ₁₋₄₂ fibers as evident by our molecular docking studies (FIG. 8A; FIG. 20). This is supported by fluorescence imaging which clearly showed the presence of smaller or breakdown fragments of Aβ fibrils following PLGA treatment (FIG. 8B, C). This is reinstated by the localization of Congo red labelled Aβ fibers in direct contact with FITC-labelled fluorescent PLGA (FIG. 8D) as well as our TEM data demonstrating the presence of PLGA nanoparticles in close alliance with Aβ fibrils (FIG. 8E). Processing of PLGA treated Aβ₁₋₄₂ fibers by DLS also revealed a shift towards the lower ordered entities compared with untreated Aβ₁₋₄₂ fibers (FIG. 8F-I) which is reinforced by the loss higher ordered entities in our Native gel analysis (FIG. 8J). Additionally, our Fluorescence quenching effect of labelled PLGA in the presence of increasing concentrations of matured Aβ₁₋₄₂ fibers showed a decrease in fluorescence intensity, indicating a direct interaction between PLGA and Aβ₁₋₄₂ fibers that may underlie the unzipping of the steric zippers in the matured Aβ₁₋₄₂ fibers yielding smaller fragments. The analysis of quenching data predicted a single binding site (n=1.05) with a Kd value of 6.95×10⁷M (FIG. 8K, FIGS. 17A, B).

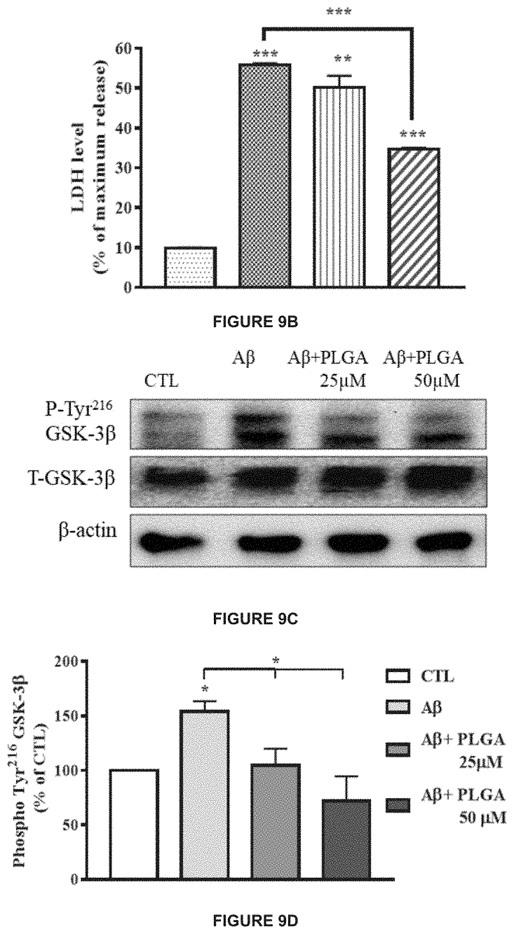

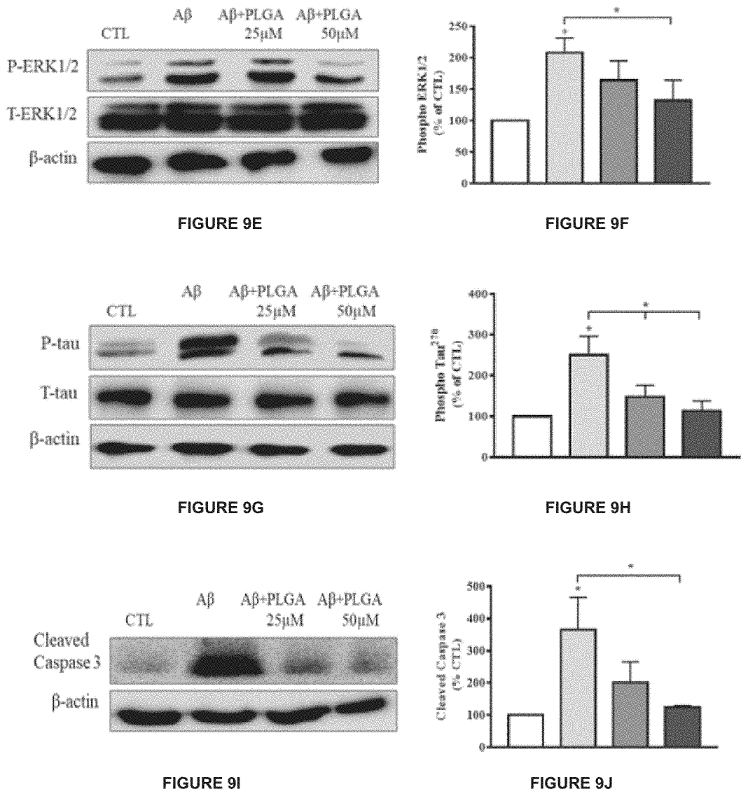

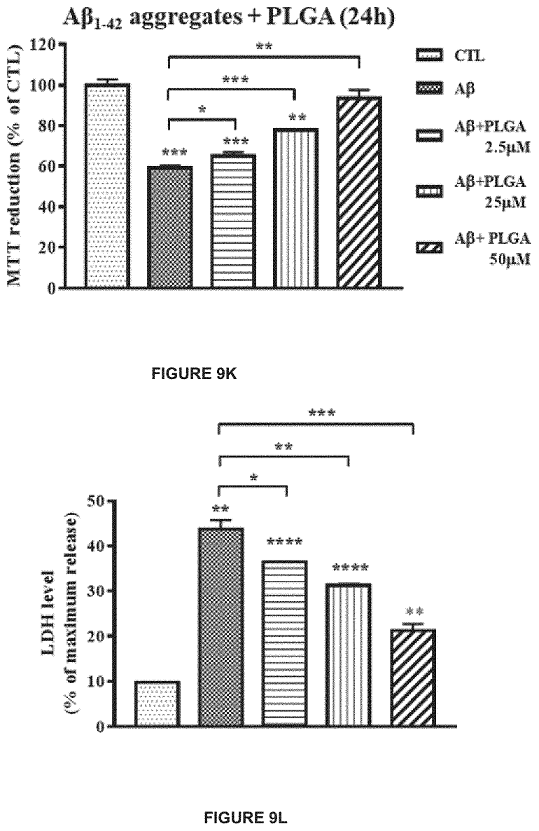

PLGA-mediated attenuation of Aβ aggregation protects cultured neurons: To evaluate if attenuation of spontaneous Aβ aggregation PLGA can influence cell viability, mouse cortical cultured neurons were exposed to Aβ samples treated with or without 25 and 50 μM PLGA. After 24 h exposure, viability of cultured neurons was assessed using MTT and LDH based cytotoxicity assays. It is evident that attenuation of Aβ aggregation by PLGA significantly increased viability of cultured neurons (FIG. 9A, B). The protective effect of PLGA-treated Aβ samples is partly mediated by attenuating activation ERK1/2 and GSK-3β, reducing phosphorylation of tau protein as well as cleavage of caspase-3— the cellular mechanisms that underlie Aβ-induced toxicity (FIG. 9C-J) (60, 103, 105, 118). To highlight the biological significance of disassembled Aβ fibers, we showed that PLGA-induced disassembly of matured Aβ fibers (i.e., after 24 h and 120 h incubation) could significantly increase neuronal viability compared to aggregated fibers (FIG. 9K, L; FIG. 21).

Effects of Aβ₁₋₄₂ on polymerized PLGA: PLGA used in the study is a co-polymer composed of 50% lactic acid and 50% glycolic acid. Since polymerization and L vs D isomeric forms can alter molecular conformation, chirality or biological properties, we wanted to determine if PLGA following interaction with Aβ is depolymerized into monomers of lactic and glycolic acids. Consequently, we carried out mass spectroscopic analysis of lactic acid, glycolic acid and PLGA with or without Aβ₁₋₄₂ treatment. Our results clearly showed that 50 μM PLGA following 120 and 240 h incubation, but not after 24 h incubation, with 10 μM Aβ₁₋₄₂ is depolymerized into glycolic acid, lactic acid and dimers of lactic acid (FIGS. 22-24).

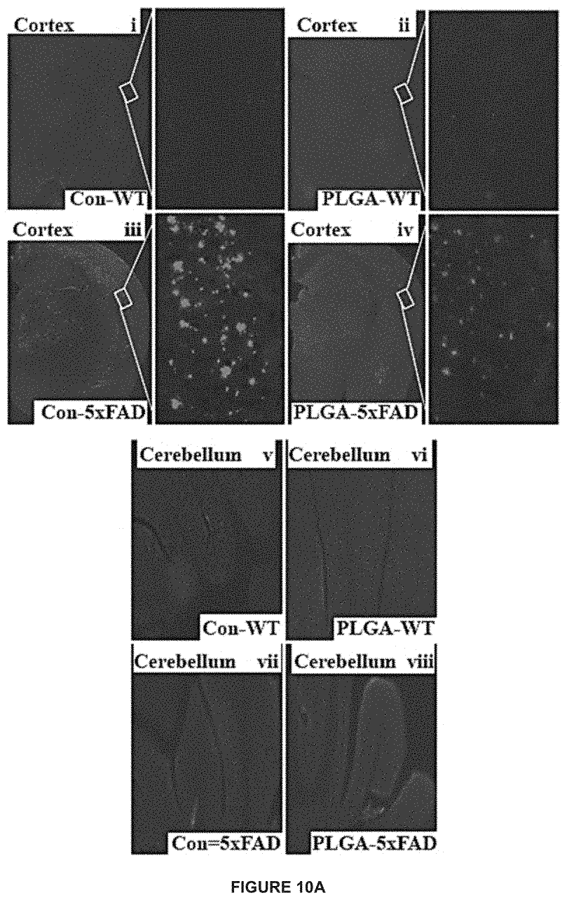

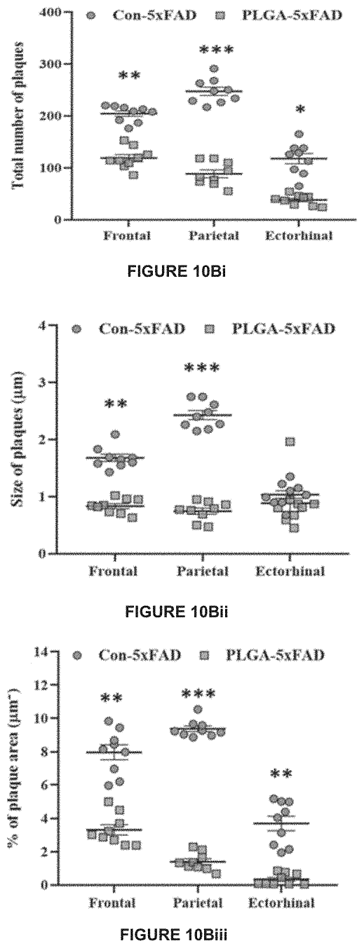

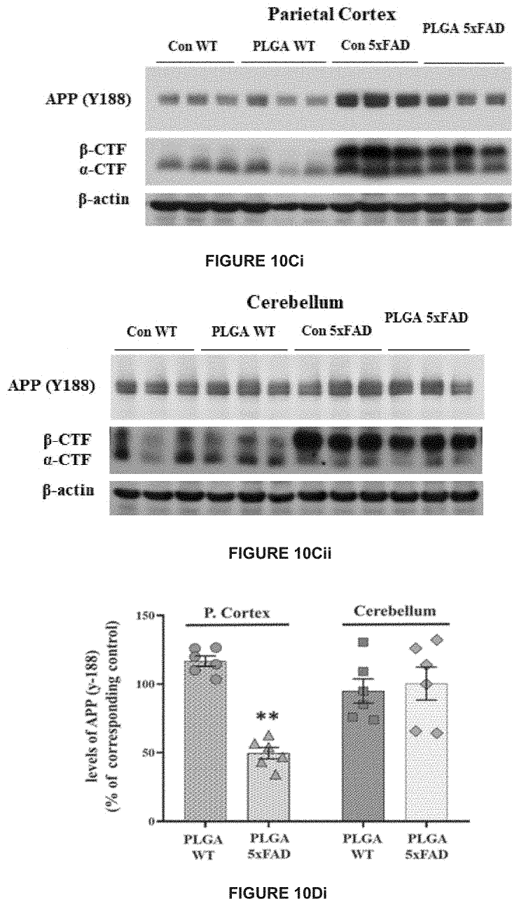

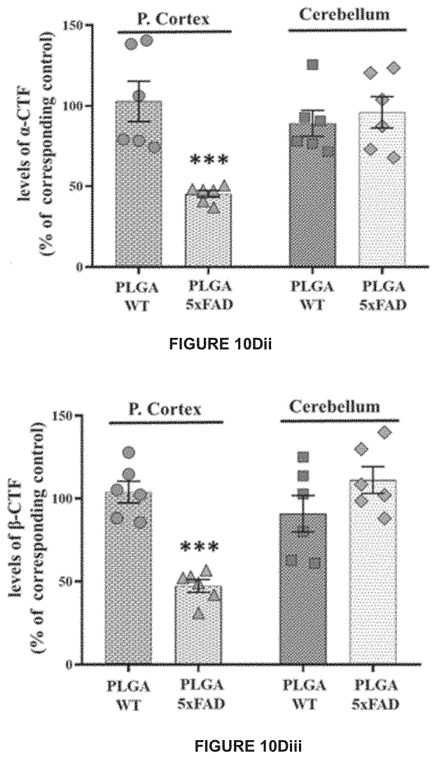

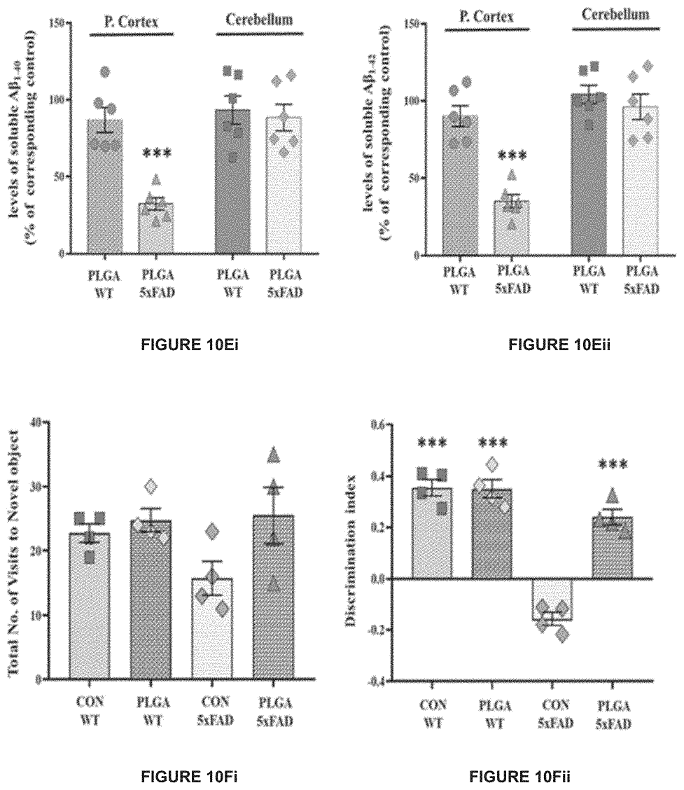

PLGA attenuates AD-related pathology in 5×FAD mice: Earlier studies reported that PLGA encapsulated drugs/agents such as memantine and curcumin exhibit satisfactory biocompatibility without any significant toxicity (34, 42, 44, 119). In keeping with these studies, chronic intracerebroventricular administration of native PLGA did not lead to fluctuations in body weight, abnormal behavior or adverse clinical signs suggesting that PLGA in the given dose is safe and without any toxicity. To examine if PLGA administration can influence Aβ pathology in 5×FAD mice, we first measured the cortical Aβ plaques load labelled with OC and 4G8 antibodies in CSF- and PLGA-treated 5×FAD mice. Our quantitative analysis revealed that the number as well as percentage of areas occupied by Aβ plaques are significantly reduced in PLGA-treated 5×FAD mice. Additionally, the average size of cortical Aβ plaques in PLGA-treated mice was smaller than the CSF-treated 5×FAD mice (FIG. 10A, B). In parallel, the levels of APP holoprotein and its cleaved products APP-CTFα and APP-CTFβ were significantly decreased in the cortex, but not in the unaffected cerebellum, of PLGA-treated 5×FAD mice. However, the levels of APP, APP-CTFα or APP-CTFβ did not differ significantly either in the cortex or in the cerebellum of CSF- and PLGA-treated WT mice (FIG. 10C, D). Accompanying the plaque load and APP holoprotein, the steady-state levels of human Aβ₁₋₄₀ and Aβ₁₋₄₂ were markedly decreased in the cortex, but not in the cerebellum, of PLGA-treated 5×FAD mice (FIG. 10E).

PLGA attenuates novel-object recognition deficits in 5×FAD mice: Memory deficits represent one of the main clinical symptoms of AD (2, 8, 120). As reported in AD patients, 5×FAD mice exhibit object recognition memory deficits starting 2.5 months of age (121). To determine if PLGA can attenuate/rescue memory deficits, we performed novel object memory test in WT and 5×FAD mice following 28 days chronic administration of the unconjugated nanoparticles. As expected, 5×FAD mice displayed impaired recognition memory, as evident by decreased discrimination index to a novel object 24 h after training with two similar objects. PLGA administered 5×FAD mice, on the other hand, spent more time on the novel object than on the familiar object, indicating that impairment of non-spatial working memory in 5×FAD mice can be attenuated by unconjugated PLGA. The memory index in WT mice, however, was not affected by PLGA administration (FIG. 10F). The effect of PLGA on 5×FAD mice was apparent in the absence of any alteration in exploratory activity between groups during the initial exposure period or between trials.

### SIGNIFICANCE

At present, there is no effective treatment to prevent/arrest the progression of AD. Over the last decade, nanoparticles have been explored extensively as an area of novel therapeutic modalities for the treatment of AD pathology. In almost all cases nanoparticles including PLGA are used in transporting drugs such as donepezil, memantine, curcumin, selegiline and the beneficial effects on cellular or animal models of AD are attributed to the drug and not the nanoparticles. Our data obtained so far strongly indicate that PLGA nanoparticles without conjugation with any drug/agent can protect and to some extent rescue cultured mouse cortical neurons against Aβ-mediated toxicity by restoring lysosomal integrity as well as regulating specific intracellular signaling cascades. We also observed that PLGA nanoparticles, by interacting with specific hydrophobic domains of the Aβ peptide, can not only inhibit spontaneous aggregation but also can trigger disassembly of aggregated Aβ fibers. Additionally, Aβ samples collected following PLGA treatment can markedly enhance neuronal viability compared to PLGA untreated Aβ samples. Finally, we revealed that intracerebroventricular administration of PLGA into 5×FAD mouse model of AD can able to attenuate cognitive deficit as well as AD-related pathology. Considering the evidence that PLGA microspheres can cross the BBB, we strongly believe that native PLGA may have a unique beneficial effect in the treatment of AD-related pathologies.

### LITERATURE CITED

1. Selkoe D J, Hardy J. The amyloid hypothesis of Alzheimer's disease at 25 years. EMBO Mol Med. 2016; 8(6):595-608.
2. Lane C A, Hardy J, Schott J M. Alzheimer's disease. Eur J Neurol.
3. Mayeux R, Stern Y. Epidemiology of Alzheimer disease. Cold Spring Harb Perspect Med. 2012; 2(8).
4. Rosenberg R N, Lambracht-Washington D, Yu G, Xia W. Genomics of Alzheimer Disease: A Review. JAMA Neurol. 2016; 73(7):867-74.
5. Bertram L, Tanzi R E. The genetics of Alzheimer's disease. Prog Mol Biol Transl Sci. 2012; 107:79-100.
6. Karch C M, Goate A M. Alzheimer's disease risk genes and mechanisms of disease pathogenesis. Biol Psychiatry. 2015; 77(1):43-51.
7. St George-Hyslop P H, Petit A. Molecular biology and genetics of Alzheimer's disease. C R Biol. 2005; 328(2):119-30.
8. Nelson P T, Braak H, Markesbery W R. Neuropathology and cognitive impairment in Alzheimer disease: a complex but coherent relationship. J Neuropathol Exp Neurol. 2009; 68(1):1-14.
9. Perl D P. Neuropathology of Alzheimer's disease. Mt Sinai J Med. 2010; 77(1):32-42.
10. Haass C, Schlossmacher M G, Hung A Y, Vigo-Pelfrey C, Mellon A, Ostaszewski B L, et al. Amyloid beta-peptide is produced by cultured cells during normal metabolism. Nature. 1992; 359(6393):322-5.
11. Seubert P, Vigo-Pelfrey C, Esch F, Lee M, Dovey H, Davis D, et al. Isolation and quantification of soluble Alzheimer's beta-peptide from biological fluids. Nature. 1992; 359(6393):325-7.
12. Dawkins E, Small D H. Insights into the physiological function of the beta-amyloid precursor protein: beyond Alzheimer's disease. J Neurochem. 2014; 129(5):756-69.
13. Muller U C, Deller T, Korte M. Not just amyloid: physiological functions of the amyloid precursor protein family. Nat Rev Neurosci. 2017; 18(5):281-98.
14. Straub J E, Thirumalai D. Toward a molecular theory of early and late events in monomer to amyloid fibril formation. Annu Rev Phys Chem. 2011; 62:437-63.
15. Chen G F, Xu T H, Yan Y, Zhou Y R, Jiang Y, Melcher K, et al. Amyloid beta: structure, biology and structure-based therapeutic development. Acta Pharmacol Sin. 2017; 38(9):1205-35.
16. Bisceglia F, Natalello A, Serafini M M, Colombo R, Verga L, Lanni C, et al. An integrated strategy to correlate aggregation state, structure and toxicity of Ass 1-42 oligomers. Talanta. 2018; 188:17-26.
17. Lee S J, Nam E, Lee H J, Savelieff M G, Lim M H. Towards an understanding of amyloid-beta oligomers: characterization, toxicity mechanisms, and inhibitors. Chem Soc Rev. 2017; 46(2):310-23.
18. Revett T J, Baker G B, Jhamandas J, Kar S. Glutamate system, amyloid ss peptides and tau protein: functional interrelationships and relevance to Alzheimer disease pathology. J Psychiatry Neurosci. 2013; 38(1):6-23.
19. Herrmann N, Chau S A, Kircanski I, Lanctot K L. Current and emerging drug treatment options for Alzheimer's disease: a systematic review. Drugs. 2011; 71(15):2031-65.
20. Anand R, Gill K D, Mandi A A. Therapeutics of Alzheimer's disease: Past, present and future. Neuropharmacology. 2014; 76 Pt A:27-50.
21. Yang P, Sun F. Aducanumab: The first targeted Alzheimer's therapy. Drug Discov Ther. 2021; 15(3):166-168.
22. Sabbagh M N. Drug development for Alzheimer's disease: where are we now and where are we headed? Am J Geriatr Pharmacother. 2009; 7(3):167-85.
23. Ghezzi L, Scarpini E, Galimberti D. Disease-modifying drugs in Alzheimer's disease. Drug Des Devel Ther. 2013; 7:1471-8.
24. Bachurin S O, Bovina E V, Ustyugov A A. Drugs in Clinical Trials for Alzheimer's Disease: The Major Trends. Med Res Rev. 2017; 37(5):1186-225.
25. Re F, Gregori M, Masserini M. Nanotechnology for neurodegenerative disorders. Nanomedicine. 2012; 8 Suppl 1:S51-8.
26. Fazil M, Shadab, Baboota S, Sahni J K, Ali J. Nanotherapeutics for Alzheimer's disease (AD): Past, present and future. J Drug Target. 2012; 20(2):97-113.
27. Krol S, Macrez R, Docagne F, Defer G, Laurent S, Rahman M, et al. Therapeutic benefits from nanoparticles: the potential significance of nanoscience in diseases with compromise to the blood brain barrier. Chem Rev. 2013; 113(3):1877-903.
28. Gao H. Progress and perspectives on targeting nanoparticles for brain drug delivery. Acta Pharm Sin B. 2016; 6(4):268-86.
29. Vinogradov S V, Bronich T K, Kabanov A V. Nanosized cationic hydrogels for drug delivery: preparation, properties and interactions with cells. Adv Drug Deliv Rev. 2002; 54(1):135-47.
30. Sun D, Li N, Zhang W, Zhao Z, Mou Z, Huang D, et al. Design of PLGA-functionalized quercetin nanoparticles for potential use in Alzheimer's disease. Colloids Surf B Biointerfaces. 2016; 148:116-29.
31. Baysal I, Ucar G, Gultekinoglu M, Ulubayram K, Yabanoglu-Ciftci S. Donepezil loaded PLGA-b-PEG nanoparticles: their ability to induce destabilization of amyloid fibrils and to cross blood brain barrier in vitro. J Neural Transm (Vienna). 2017; 124(1):33-45.
32. Fornaguera C, Feiner-Gracia N, Caldero G, Garcia-Celma M J, Solans C. Galantamine-loaded PLGA nanoparticles, from nano-emulsion templating, as novel advanced drug delivery systems to treat neurodegenerative diseases. Nanoscale. 2015; 7(28):12076-84.
33. Silva-Abreu M, Calpena A C, Andres-Benito P, Aso E, Romero I A, Roig-Carles D, et al. PPARgamma agonist-loaded PLGA-PEG nanocarriers as a potential treatment for Alzheimer's disease: in vitro and in vivo studies. Int J Nanomedicine. 2018; 13:5577-90.
34. Fan S, Zheng Y, Liu X, Fang W, Chen X, Liao W, et al. Curcumin-loaded PLGA-PEG nanoparticles conjugated with B6 peptide for potential use in Alzheimer's disease. Drug Deliv. 2018; 25(1):1091-102.
35. Sharma S, Nehru B, Saini A. Inhibition of Alzheimer's amyloid-beta aggregation in-vitro by carbenoxolone: Insight into mechanism of action. Neurochem Int. 2017; 108:481-93.
36. Baysal I, Yabanoglu-Ciftci S, Tunc-Sarisozen Y, Ulubayram K, Ucar G. Interaction of selegiline-loaded PLGA-b-PEG nanoparticles with beta-amyloid fibrils. J Neural Transm (Vienna). 2013; 120(6):903-10.
37. Ashraf G M, Tabrez S, Jabir N R, Firoz C K, Ahmad S, Hassan I, et al. An Overview on Global Trends in Nanotechnological Approaches for Alzheimer Therapy. Curr Drug Metab. 2015; 16(8):719-27.
38. Sahni J K, Doggui S, Ali J, Baboota S, Dao L, Ramassamy C. Neurotherapeutic applications of nanoparticles in Alzheimer's disease. J Control Release. 2011; 152(2):208-31.
39. Ansari S A, Satar R, Perveen A, Ashraf G M. Current opinion in Alzheimer's disease therapy by nanotechnology-based approaches. Curr Opin Psychiatry. 2017; 30(2):128-35.
40. Hajipour M J, Santoso M R, Rezaee F, Aghaverdi H, Mahmoudi M, Perry G. Advances in Alzheimer's Diagnosis and Therapy: The Implications of Nanotechnology. Trends Biotechnol. 2017; 35(10):937-53.
41. Aalinkeel R, Kutscher H L, Singh A, Cwiklinski K, Khechen N, Schwartz S A, et al. Neuroprotective effects of a biodegradable poly(lactic-co-glycolic acid)-ginsenoside Rg3 nanoformulation: a potential nanotherapy for Alzheimer's disease? J Drug Target. 2018; 26(2):182-93.
42. Sanchez-Lopez E, Ettcheto M, Egea M A, Espina M, Cano A, Calpena A C, et al. Memantine loaded PLGA PEGylated nanoparticles for Alzheimer's disease: in vitro and in vivo characterization. J Nanobiotechnology. 2018; 16(1):32.
43. Jeon S G, Cha M Y, Kim J I, Hwang T W, Kim K A, Kim T H, et al. Vitamin D-binding protein-loaded PLGA nanoparticles suppress Alzheimer's disease-related pathology in SXFAD mice. Nanomedicine. 2019; 17:297-307.
44. Huo X, Zhang Y, Jin X, Li Y, Zhang L. A novel synthesis of selenium nanoparticles encapsulated PLGA nanospheres with curcumin molecules for the inhibition of amyloid beta aggregation in Alzheimer's disease. J Photochem Photobiol B. 2019; 190:98-102.
45. Anand B G, Dubey K, Shekhawat D S, Kar K. Capsaicin-Coated Silver Nanoparticles Inhibit Amyloid Fibril Formation of Serum Albumin. Biochemistry. 2016; 55(24):3345-8.
46. Assarsson A, Hellstrand E, Cabaleiro-Lago C, Linse S. Charge dependent retardation of amyloid beta aggregation by hydrophilic proteins. ACS Chem Neurosci. 2014; 5(4):266-74.
47. Liu H, Ojha B, Morris C, Jiang M, Wojcikiewicz E P, Rao P P, et al. Positively Charged Chitosan and N-Trimethyl Chitosan Inhibit Abeta40 Fibrillogenesis. Biomacromolecules. 2015; 16(8):2363-73.
48. Gobbi M, Re F, Canovi M, Beeg M, Gregori M, Sesana S, et al. Lipid-based nanoparticles with high binding affinity for amyloid-beta1-42 peptide. Biomaterials. 2010; 31(25):6519-29.
49. Taylor M, Moore S, Mourtas S, Niarakis A, Re F, Zona C, et al. Effect of curcumin-associated and lipid ligand-functionalized nanoliposomes on aggregation of the Alzheimer's Abeta peptide. Nanomedicine. 2011; 7(5):541-50.
50. Astete C E, Sabliov C M. Synthesis and characterization of PLGA nanoparticles. J Biomater Sci Polym Ed. 2006; 17(3):247-89.
51. Makadia H K, Siegel S J. Poly Lactic-co-Glycolic Acid (PLGA) as Biodegradable Controlled Drug Delivery Carrier. Polymers (Basel). 2011; 3(3):1377-97.
52. Wang Y, Qu W, Choi S H (2016) FDA's regulatory science program for generic PLA/PLGA-based drug program. Am. Pharmaceu. Rev. 2016; 20:4.
53. Jain R A. The manufacturing techniques of various drug loaded biodegradable poly(lactide-co-glycolide) (PLGA) devices. Biomaterials. 2000; 21(23):2475-90.
54. Kitchell J P, Wise D L. Poly(lactic/glycolic acid) biodegradable drug-polymer matrix systems. Methods Enzymol. 1985; 112:436-48.
55. Shive M S, Anderson J M. Biodegradation and biocompatibility of PLA and PLGA microspheres. Adv Drug Deliv Rev. 1997; 28(1):5-24.
56. Nguyen K T, Pham M N, Vo T V, Duan W, Tran P H, Tran T T. Strategies of Engineering Nanoparticles for Treating Neurodegenerative Disorders. Curr Drug Metab. 2017; 18(9):786-97.
57. Athanasiou K A, Niederauer G G, Agrawal C M. Sterilization, toxicity, biocompatibility and clinical applications of polylactic acid/polyglycolic acid copolymers. Biomaterials. 1996; 17(2):93-102.
58. Baltazar G C, Guha S, Lu W, Lim J, Boesze-Battaglia K, Laties A M, et al. Acidic nanoparticles are trafficked to lysosomes and restore an acidic lysosomal pH and degradative function to compromised ARPE-19 cells. PLoS One. 2012; 7(12):e49635.
59. Bourdenx M, Daniel J, Genin E, Soria F N, Blanchard-Desce M, Bezard E, et al. Nanoparticles restore lysosomal acidification defects: Implications for Parkinson and other lysosomal-related diseases. Autophagy. 2016; 12(3):472-83.
60. Foroutanpay B V, Kumar J, Kang S G, Danaei N, Westaway D, Sim V L, et al. The Effects of N-terminal Mutations on beta-amyloid Peptide Aggregation and Toxicity. Neuroscience. 2018; 379:177-88.
61. Amritraj A, Wang Y, Revett T J, Vergote D, Westaway D, Kar S. Role of cathepsin D in U18666A-induced neuronal cell death: potential implication in Niemann-Pick type C disease pathogenesis. J Biol Chem. 2013; 288(5):3136-52.
62. Wang Y, Buggia-Prevot V, Zavorka M E, Bleackley R C, MacDonald R G, Thinakaran G, et al. Overexpression of the Insulin-Like Growth Factor I I Receptor Increases beta-Amyloid Production and Affects Cell Viability. Mol Cell Biol. 2015; 35(14):2368-84.
63. Wu Q, Li Z, Mellor P, Zhou Y, Anderson D H, Liu Q. The role of PTEN-HCV core interaction in hepatitis C virus replication. Sci Rep. 2017; 7(1):3695.
64. Anand B G, Prajapati K P, Shekhawat D S, Kar K. Tyrosine-Generated Nanostructures Initiate Amyloid Cross-Seeding in Proteins Leading to a Lethal Aggregation Trap. Biochemistry. 2018; 57(35):5202-9.
65. Anand B G, Prajapati K P, Kar K. Abetal-40 mediated aggregation of proteins and metabolites unveils the relevance of amyloid cross-seeding in amyloidogenesis. Biochem Biophys Res Commun. 2018; 501(1):158-64.
66. Anand B G, Dubey K, Shekhawat D S, Kar K. Intrinsic property of phenylalanine to trigger protein aggregation and hemolysis has a direct relevance to phenylketonuria. Sci Rep. 2017; 7(1):11146.
67. Anand B G, Prajapati K P, Dubey K, Ahamad N, Shekhawat D S, Rath P C, et al. Self-Assembly of Artificial Sweetener Aspartame Yields Amyloid-like Cytotoxic Nanostructures. ACS Nano. 2019; 13(5):6033-49.
68. Dubey K, Anand B G, Shekhawat D S, Kar K. Eugenol prevents amyloid formation of proteins and inhibits amyloid-induced hemolysis. Sci Rep. 2017; 7:40744.
69. Anand B G, Dubey K, Shekhawat D S, Prajapati K P, Kar K. Strategically Designed Antifibrotic Gold Nanoparticles to Prevent Collagen Fibril Formation. Langmuir. 2017; 33(46):13252-61.
70. Dallakyan S, Olson A J. Small-Molecule Library Screening by Docking with PyRx. Methods Mol Biol. 2015; 1263:243-50.
71. Pettersen E F, Goddard T D, Huang C C, Couch G S, Greenblatt D M, Meng E C, et al. UCSF Chimera—a visualization system for exploratory research and analysis. J Comput Chem. 2004; 25(13):1605-12.
72. Dahiya V, Anand B G, Kar K, Pal S. In vitro interaction of organophosphate metabolites with bovine serum albumin: A comparative 1H NMR, fluorescence and molecular docking analysis. Pestic Biochem Physiol. 2019.
73. Anand B G, Shekhawat D S, Dubey K, Kar K. Uniform, Polycrystalline, and Thermostable Piperine-Coated Gold Nanoparticles to Target Insulin Fibril Assembly. ACS Biomaterials Science & Engineering. 2017; 3(6):1136-45.
74. Lakowicz J R, Weber G. Quenching of fluorescence by oxygen. A probe for structural fluctuations in macromolecules. Biochemistry. 1973; 12(21):4161-70.
75. Lu R, Li W W, Katzir A, Raichlin Y, Yu H Q, Mizaikoff B. Probing the secondary structure of bovine serum albumin during heat-induced denaturation using mid-infrared fiberoptic sensors. Analyst. 2015; 140(3):765-70.
76. Wetzel R, Chemuru S, Misra P, Kodali R, Mukherjee S, Kar K. An Aggregate Weight-Normalized Thioflavin-T Measurement Scale for Characterizing Polymorphic Amyloids and Assembly Intermediates. Methods Mol Biol. 2018; 1777:121-44.
77. Prajapati K P, Singh A P, Dubey K, Ansari M, Temgire M, Anand B G, et al. Myricetin inhibits amyloid fibril formation of globular proteins by stabilizing the native structures. Colloids Surf B Biointerfaces. 2019; 186:110640.
78. Dubey K, Anand B G, Badhwar R, Bagler G, Navya P N, Daima H K, et al. Tyrosine- and tryptophan-coated gold nanoparticles inhibit amyloid aggregation of insulin. Amino Acids. 2015; 47(12):2551-60.
79. Li X, Zhang X, Ladiwala A R, Du D, Yadav J K, Tessier P M, et al. Mechanisms of transthyretin inhibition of beta-amyloid aggregation in vitro. J Neurosci. 2013; 33(50):19423-33.
80. Oakley H, Cole S L, Logan S, Maus E, Shao P, Craft J, et al. Intraneuronal beta-amyloid aggregates, neurodegeneration, and neuron loss in transgenic mice with five familial Alzheimer's disease mutations: potential factors in amyloid plaque formation. J Neurosci. 2006; 26(40):10129-40.
81. Wang Y, Wu Q, Anand B G, Karthivashan G, Phukan G, Yang J, et al. Significance of cytosolic cathepsin D in Alzheimer's disease pathology: Protective cellular effects of unconjugated nanoparticles against β-amyloid toxicity. Neuropathol Appl Neurobiol (in press). 2020.
82. Maulik M, Peake K, Chung J, Wang Y, Vance J E, Kar S. APP overexpression in the absence of NPC1 exacerbates metabolism of amyloidogenic proteins of Alzheimer's disease. Hum Mol Genet. 2015; 24(24):7132-50.
83. Song M S, Baker G B, Todd K G, Kar S. Inhibition of beta-amyloid1-42 internalization attenuates neuronal death by stabilizing the endosomal-lysosomal system in rat cortical cultured neurons. Neuroscience. 2011; 178:181-8.
84. Kodam A, Ourdev D, Maulik M, Hariharakrishnan J, Banerjee M, Wang Y, et al. A role for astrocyte-derived amyloid beta peptides in the degeneration of neurons in an animal model of temporal lobe epilepsy. Brain Pathol. 2019; 29(1):28-44.
85. Chung J, Phukan G, Vergote D, Mohamed A, Maulik M, Stahn M, et al. Endosomal-Lysosomal Cholesterol Sequestration by U18666A Differentially Regulates Amyloid Precursor Protein (APP) Metabolism in Normal and APP-Overexpressing Cells. Mol Cell Biol. 2018; 38(11).
86. Maulik M, Ghoshal B, Kim J, Wang Y, Yang J, Westaway D, et al. Mutant human APP exacerbates pathology in a mouse model of NPC and its reversal by a beta-cyclodextrin. Hum Mol Genet. 2012; 21(22):4857-75.
87. Reiss A B, Arain H A, Stecker M M, Siegart N M, Kasselman L J. Amyloid toxicity in Alzheimer's disease. Rev Neurosci. 2018; 29(6):613-27.
88. Wang J, Dickson D W, Trojanowski J Q, Lee V M. The levels of soluble versus insoluble brain Abeta distinguish Alzheimer's disease from normal and pathologic aging. Exp Neurol. 1999; 158(2):328-37.
89. Mawuenyega K G, Sigurdson W, Ovod V, Munsell L, Kasten T, Morris J C, et al. Decreased clearance of CNS beta-amyloid in Alzheimer's disease. Science. 2010; 330(6012):1774.
90. Kayed R, Lasagna-Reeves C A. Molecular mechanisms of amyloid oligomers toxicity. J Alzheimers Dis. 2013; 33 Suppl 1:S67-78.
91. Roberg K, Ollinger K. Oxidative stress causes relocation of the lysosomal enzyme cathepsin D with ensuing apoptosis in neonatal rat cardiomyocytes. Am J Pathol. 1998; 152(5):1151-6.
92. Ditaranto K, Tekirian T L, Yang A J. Lysosomal membrane damage in soluble Abeta-mediated cell death in Alzheimer's disease. Neurobiol Dis. 2001; 8(1):19-31.
93. Gowran A, Campbell V A. A role for p53 in the regulation of lysosomal permeability by delta 9-tetrahydrocannabinol in rat cortical neurones: implications for neurodegeneration. J Neurochem. 2008; 105(4):1513-24.
94. Amritraj A, Peake K, Kodam A, Salio C, Merighi A, Vance J E, et al. Increased activity and altered subcellular distribution of lysosomal enzymes determine neuronal vulnerability in Niemann-Pick type C1-deficient mice. Am J Pathol. 2009; 175(6):2540-56.
95. Umeda T, Tomiyama T, Sakama N, Tanaka S, Lambert M P, Klein W L, et al. Intraneuronal amyloid beta oligomers cause cell death via endoplasmic reticulum stress, endosomal/lysosomal leakage, and mitochondrial dysfunction in vivo. J Neurosci Res. 2011; 89(7):1031-42.
96. Youmans K L, Tai L M, Kanekiyo T, Stine W B, Jr., Michon S C, Nwabuisi-Heath E, et al. Intraneuronal Abeta detection in 5×FAD mice by a new Abeta-specific antibody. Mol Neurodegener. 2012; 7:8.
97. Wang F, Gomez-Sintes R, Boya P. Lysosomal membrane permeabilization and cell death. Traffic. 2018; 19(12):918-31.
98. Oberst A, Bender C, Green D R. Living with death: the evolution of the mitochondrial pathway of apoptosis in animals. Cell Death Differ. 2008; 15(7):1139-46.
99. Stoka V, Turk V, Turk B. Lysosomal cathepsins and their regulation in aging and neurodegeneration. Ageing Res Rev. 2016; 32:22-37.
100. Banerjee M, Sasse V A, Wang Y, Maulik M, Kar S. Increased levels and activity of cathepsins B and D in kainate-induced toxicity. Neuroscience. 2015; 284:360-73.
101. Fraldi A, Klein A D, Medina D L, Settembre C. Brain Disorders Due to Lysosomal Dysfunction. Annu Rev Neurosci. 2016; 39:277-95.
102. Repnik U, Turk B. Lysosomal-mitochondrial cross-talk during cell death. Mitochondrion. 2010; 10(6):662-9.
103. Wei Z, Song M S, MacTavish D, Jhamandas J H, Kar S. Role of calpain and caspase in beta-amyloid-induced cell death in rat primary septal cultured neurons. Neuropharmacology. 2008; 54(4):721-33.
104. Liu H, Xie B, Dong X, Zhang L, Wang Y, Liu F, et al. Negatively charged hydrophobic nanoparticles inhibit amyloid β-protein fibrillation: The presence of an optimal charge density. React Funct Polym. 2016; 103:108-16.
105. Song M S, Rauw G, Baker G B, Kar S. Memantine protects rat cortical cultured neurons against beta-amyloid-induced toxicity by attenuating tau phosphorylation. Eur J Neurosci. 2008; 28(10):1989-2002.
106. Ntsapi C, Lumkwana D, Swart C, du Toit A, Loos B. New Insights Into Autophagy Dysfunction Related to Amyloid Beta Toxicity and Neuropathology in Alzheimer's Disease. Int Rev Cell Mol Biol. 2018; 336:321-61.
107. Wolfe D M, Lee J H, Kumar A, Lee S, Orenstein S J, Nixon R A. Autophagy failure in Alzheimer's disease and the role of defective lysosomal acidification. Eur J Neurosci. 2013; 37(12):1949-61.
108. Kumar E K, Haque N, Prabhu N P. Kinetics of protein fibril formation: Methods and mechanisms. Int J Biol Macromol. 2017; 100:3-10.
109. Galzitskaya O V, Galushko El, Selivanova O M. Studies of the Process of Amyloid Formation by Abeta Peptide. Biochemistry (Mosc). 2018; 83(Suppl 1):562-580.
110. Arosio P, Knowles T P, Linse S. On the lag phase in amyloid fibril formation. Phys Chem Chem Phys. 2015; 17(12):7606-18.
111. Hawkes C A, Ng V, McLaurin J. Small molecule inhibitors of Aβ-aggregation and neurotoxicity. Drug Dev Res. 2009; 70:111-24.
112. Zhang X, Fu Z, Meng L, He M, Zhang Z. The Early Events That Initiate beta-Amyloid Aggregation in Alzheimer's Disease. Front Aging Neurosci. 2018; 10:359.
113. Man B Y, Chan H, Leung C, Chan D S, Bai L, Jiang Z H, et al. Group 9 metal-based inhibitors of β-amyloid (1-40) fibrillation as potential therapeutic agents for Alzheimer's disease. Chem Sci. 2011; 2:917-21.
114. Funke S A, Willbold D. Peptides for therapy and diagnosis of Alzheimer's disease. Curr Pharm Des. 2012; 18(6):755-67.
115. Panza F, Lozupone M, Logroscino G, Imbimbo B P. A critical appraisal of amyloid-beta-targeting therapies for Alzheimer disease. Nat Rev Neurol. 2019; 15(2):73-88.
116. Moran S D, Zhang T O, Decatur S M, Zanni M T. Amyloid fiber formation in human gammaD-Crystallin induced by U V-B photodamage. Biochemistry. 2013; 52(36):6169-81
117. Van Nostrand W E, Melchor J P, Cho H S, Greenberg S M, Rebeck G W. Pathogenic effects of D23N Iowa mutant amyloid beta-protein. J Biol Chem. 2001; 276(35):32860-6.
118. Donida B, Jacques C E D, Mescka C P, Rodrigues D G B, Marchetti D P, Ribas G, et al. Oxidative damage and redox in Lysosomal Storage Disorders: Biochemical markers. Clin Chim Acta. 2017; 466:46-53.
119. Huang N, Lu S, Liu X G, Zhu J, Wang Y J, Liu R T. PLGA nanoparticles modified with a BBB-penetrating peptide co-delivering Abeta generation inhibitor and curcumin attenuate memory deficits and neuropathology in Alzheimer's disease mice. Oncotarget. 2017; 8(46):81001-13.
120. Chen X Q, Mobley W C. Alzheimer Disease Pathogenesis: Insights From Molecular and Cellular Biology Studies of Oligomeric Abeta and Tau Species. Front Neurosci. 2019; 13:659.
121. Kosel F, Torres Munoz P, Yang J R, Wong A A, Franklin T B. Age-related changes in social behaviours in the 5×FAD mouse model of Alzheimer's disease. Behav Brain Res. 2019; 362:160-172.

TABLE 1
Details of the primary antibodies used in this study

| | | IF | WB/DB | |
|---|---|---|---|---|
| Antibody Type | Type | dilution | dilution | Source |
| Apoptosis-inducing | Polyclonal | N/A | 1:500 | Santa Cruz |
| factor (AIF) | | | | |
| Autophagy protein 5 | Polyclonal | N/A | 1:1000 | EMD Millipore |
| (ATG5) | | | | |
| Amyloic precursor | Monoclonal | NA | 1:5000 | Abcam |
| protein (APP; clone | | | | |
| Y188) | | | | |
| Amyloid Fibrils OC | Polyclonal | 1:500 | 1:1000 | Sigma-Aldrich |
| Beclin 1 | Monoclonal | NA | 1:200 | Santa Cruz |
| Cleaved-Caspase-3 | Monoclonal | NA | 1:1000 | Cell Signaling |
| Phospho-ERK | Polyclonal | NA | 1:1000 | Cell Signaling |
| Total-ERK | Monoclonal | N/A | 1:1000 | Cell Signaling |
| Phospho-GSK | Polyclonal | N/A | 1:1000 | Abeam. |
| Total-GSK | Monoclonal | N/A | 1:1000 | Abeam |
| LAMP1 | Polyclonal | 1:1000 | NA | Abcam |
| Microtubule-associated | Polyclonal | NA | 1:3000 | MBL |
| protein light chain 3 | | | | |
| (LC3) | | | | |
| Sequestosome-1 (P62) | Monoclonal | NA | 1:1000 | EMD Millipore |
| Tau (AT180) | Monoclonal | NA | 1:1000 | Thermo Fisher |
| | | | | Scientific |
| Tau (AT270) | Monoclonal | NA | 1:1000 | Thermo Fisher |
| | | | | Scientific |
| β-actin | Monoclonal | NA | 1:5000 | Sigma-Aldrich |
| β-amyloid, 1-15 (3A1) | Monoclonal | NA | 1:1000 | BioLegend |
| β-amyloid, 1-16 (6E10) | Monoclonal | NA | 1:1000 | BioLegend |
| β-amyloid, 17-24 (4G8) | Monoclonal | NA | 1:1000 | BioLegend |
| β-amyloid, 23-29 | Polyclonal | NA | 1:1000 | Anaspec |
| β-amyloid 37-42 | Polyclonal | NA | 1:1000 | Antibodies- |
| | | | | online |

IF: immunofluorescence; WB: western blotting; DB: dot blotting; N/A: not used in that specific application.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A method of treating a subject having Alzheimer's Disease or a tauopathy or suspected of having Alzheimer's Disease or a tauopathy, comprising: administering a therapeutically effective amount of poly(D, L-lactide-co-glycolide) (PLGA).

2. The method of claim 1, wherein the poly(D,L-lactide-co-glycolide) polymer contains a 50:50 ratio of lactic and glycolic acids.

3. The method of claim 1, wherein said Alzheimer's Disease is preclinical Alzheimer's Disease, Mild cognitive impairment, or Alzheimer's dementia.

4. The method of claim 1, wherein said tauopathy is frontotemporal dementia, Pick's disease, epilepsy, or chronic traumatic encephalopathy.

5. The method of claim 1, wherein said administration comprises intracerebroventricular, intravenous, intranasal, or subcutaneous administration.

6. A method of treating a subject having Alzheimer's Disease or a tauopathy or suspected of having Alzheimer's Disease or a tauopathy, consisting of or essentially consisting of: administering a therapeutically effective amount of poly(D, L-lactide-co-glycolide) (PLGA).

7. The method of claim 6, wherein the poly(D,L-lactide-co-glycolide) polymer contains a 50:50 ratio of lactic and glycolic acids.

8. The method of claim 6, wherein said Alzheimer's Disease is preclinical Alzheimer's Disease, Mild cognitive impairment, or Alzheimer's dementia.

9. The method of claim 6, wherein said tauopathy is frontotemporal dementia, Pick's disease, epilepsy, or chronic traumatic encephalopathy.

10. The method of claim 6, wherein said administration comprises intracerebroventricular, intravenous, intranasal, or subcutaneous administration.
